(19) United States
(12) Patent Application Publication
(10) Pub. No.: US 2006/0166191 A1
(43) Pub. Date: Jul. 27, 2006

(54) Inhibition of AP-3/Gag interactions in the treatment of human immunodeficiency virus infections

(75) Inventors: Paul Spearman, Decatur, GA (US); Xinhong Dong, Nashville, TN (US); Aaron Derdowski, Nashville, TN (US)

Correspondence Address: FULBRIGHT & JAWORSKI L.L.P., 600 CONGRESS AVE., SUITE 2400, AUSTIN, TX, 78701, US

(73) Assignee: Vanderbilt University

(21) Appl. No.: 11/253,059

(22) Filed: Oct. 18, 2005

Related U.S. Application Data
(60) Provisional application No. 60/619,682, filed on Oct. 18, 2004.

(51) Int. Cl. C12Q 1/70

(52) U.S. Cl. 435/5

(57) ABSTRACT

The present invention provides for methods of identifying potential inhibitors of HIV infection and replication. More specifically, the invention identifies complexes between the δ subunit of AP-3 and Gag, which facilitate HIV assembly. Screening for inhibitors of this interaction will identify lead compounds for the treatment of HIV and AIDS.

This application claims benefit of priority to U.S. Provisional Application Ser. No. 60/619,682, filed Oct. 18, 2004, the entire contents of which are hereby incorporated by reference.

The government owns rights in the invention pursuant to funding from the National Institutes of Health (NIH RO1 AI40338 and NIH R21 AI055441).

### BACKGROUND OF THE INVENTION

1. Field of the Invention

The present invention relates generally to the fields of infectious disease and and molecular biology. More particularly, it concerns the identification of a protein complex, comprising the δ subunit of AP-3 and Gag, that is required for HIV assembly. Specifically, the invention provides method for screening of inhibitors of HIV assembly and the use of such inhibitors in the treatment of HIV and AIDS.

2. Description of Related Art

The process of retroviral particle assembly is directed by the Gag polyprotein (Freed, 1998; Wills and Craven, 1991). Gag proteins expressed in the absence of any other viral components are capable of eliciting the formation of virus-like particles (pseudovirions) of the authentic size, density, and morphology of the infectious virion. HIV Gag proteins are cleaved by the viral protease during the budding process into four structural proteins that perform unique functions in the mature virion (MA, CA, NC and p6, listed from amino- to carboxy-terminus). During assembly, the uncleaved Gag polyprotein interacts with the viral RNA and the envelope glycoprotein complex (Env) to coordinate the production of infectious virions. Gag interacts with a number of cellular factors that play an essential role in particle budding as it makes its way to the plasma membrane assembly site (Luban, 2001; Martin-Serrano et al., 2003; Strack et al., 2003; von Schwedler et al., 2003).

HIV budding occurs predominantly at the plasma membrane in T-lymphocytes and most transformed epithelial cell lines (Freed, 1998; Ono and Freed, 2004; Spearman et al., 1994). Pr55^{Gag} is synthesized on free cytosolic ribosomes, and trafficks to the plasma membrane assembly site by pathways that are not yet completely defined. The discovery that Gag interacts with TSG101 and other components of the vacuolar protein sorting (Vps) pathway has led to new insights into the route taken by Gag in the cell (Garrus et al., 2001; VerPlank et al., 2001). TSG101 is a component of the ESCRT-I complex and plays a key role in the biogenesis of multivesicular bodies (MVBs) (Babst et al., 2000; Katzmann et al., 2001). Gag binds directly to the UEV domain of TSG101 through a tetrapeptide (PTAP) domain located in the carboxyl-terminal p6 domain. In this manner, Gag acts as an Hrs homologue, and diverts TSG101 and other ESCRT components from their role in MVB formation to assist in the late steps of particle assembly (Pomillos et al., 2003).

In some cells, HIV particle budding occurs directly into the MVB rather than from the plasma membrane. This phenomenon is prominent in primary macrophages, where it appears to be the major productive pathway involved in particle formation and release (Nguyen et al., 2003; Ono and Freed, 2004; Raposo et al., 2002). Some investigators have postulated that budding into late endosomes is important in most cell types, and have suggested that Gag acts as a cargo molecule for normal cellular exocytic machinery (the “Trojan exosome” hypothesis) (Gould et al., 2003). While the role of HIV budding into endosomes in most relevant cell types such as primary T cells is debated, it is clear that Gag and MVB markers colocalize in a wide variety of cells (Nydegger et al., 2003; Sherer et al., 2003). It is likely that specific cellular trafficking machinery is responsible for bringing Gag to the MVB.

The role of the matrix (MA) region of HIV Gag in trafficking and particle assembly has been enigmatic. MA is required for incorporation of the intact HIV envelope glycoprotein (Env) into virions, as small deletions or substitutions can disrupt this incorporation and eliminate particle infectivity (Freed and Martin, 1996; Lodge et al., 1994; Yu et al., 1992). Myristylation of MA is essential for membrane interactions and for particle assembly (Bryant and Ratner, 1990; Gottlinger et al., 1989). The leading model for the function of MA in membrane binding proposes that both myristic acid and a cluster of basic residues within MA contribute to plasma membrane binding (Zhou and Resh, 1996), and this model is supported by structural data placing the myristyl anchor and the basic cluster on one membrane-binding face of the MA trimer (Hill et al., 1996; Matthews et al., 1994). However, the myristylation sequence of v-src can rescue efficient particle formation in the absence of MA, although truncation of the Env cytoplasmic tail is required for incorporation and virion infectivity in this context (Reil et al., 1998). One interpretation of this finding is that much of MA plays no functional role in assembly. However, it is perhaps more likely that the v-src sequence provides independent plasma membrane targeting information and bypasses the need for targeting information intrinsic to MA. Further support for a trafficking role of MA comes from evidence that large deletions of MA that preserve the Gag myristylation-acceptor sequence lead to the formation of particles at intracellular membranes, suggesting that a targeting signal within MA has been removed (Facke et al., 1993; Spearman et al., 1994; Yuan et al., 1993). More subtle substitutions within MA have also resulted in a shift from plasma membrane assembly to assembly within intracellular membrane compartments (Freed et al., 1994; Ono et al., 2000). Together, these findings indicate that MA plays an important role in the intracellular trafficking of Gag. However, in order to better understand the process of HIV particle assembly, it will be necessary to identify the specific cellular factors responsible for the trafficking of Gag within the cell.

### SUMMARY OF THE INVENTION

Thus, in accordance with the present invention, there is provided a method of screening a first substance for anti-HIV activity comprising (a) providing δ subunit of adaptor protein-3 (δAP-3) and Gag under conditions permitting the formation of an δAP-3/Gag complex; (b) contacting the first substance with δAP-3 and Gag; and (c) assessing the formation of δAP-3/Gag complex; wherein a decrease in the amount of δAP-3/Gag complex, as compared to δAP-3/Gag complex formed in the absence of the first substance, indicates that the first substance possesses anti-HIV activity.

Step (a) may comprise providing a cell that expresses δAP-3 and Gag, and further, comprise providing a cell infected with HIV. Alternatively, Step (a) may comprise providing δAP-3 and Gag in a cell free environment. Step (c) may comprise a two-hybrid screen, a Western blot, a a band shift assay, a sandwich ELISA, or co-immunoprecipitation. The method may further comprise performing a control reaction wherein a known inhibitor of δAP-3/Gag complex formation is used, and/or further comprising performing a control reaction wherein no inhibitor of δAP-3/Gag complex formation is used. The first substance may be a protein or peptide, a nucleic acid, such as an antisense molecule, a ribozyme or a small interfering RNA. The first substance also may be a small molecule. The method may also further comprise the addition of a second substance distinct from the first substance.

In another embodiment, there is provided a method of screening a first substance for anti-HIV activity comprising (a) providing a cell that expresses δ subunit of adaptor protein-3 (δAP-3); (b) contacting the cell with the first substance; and (c) assessing the expression of δAP-3; wherein a decrease in the amount of δAP-3, as compared to the δAP-3 expressed in the absence of the first substance, indicates that the first substance possesses anti-HIV activity.

The method may further comprise assessing the effect of the first substance on δAP-3/Gag complex formation, and/or assessing the effect of the first substance on HIV particle formation. Assessing may comprise immunoblot, ELISA, RIA, radioimmunepreciptation or quantitive RT-PCR. Alternatively, it may involve measuring release of infectious units into culture supernatant. The first substance may be a protein, a peptide, a nucleic acid or a small molecule.

In yet another embodiment, there is provided a method of inhibiting HIV infection comprising contacting a subject infected or suspected of being infected with HIV with a substance that inhibits formation of δ subunit of AP-3(δAP-3)/Gag complex. The substance may be an δAP-3 antisense molecule, an δAP-3 siRNA molecule, an anti-δAP-3 antibody molecule, a dominant negative form of δAP-3, or an expression construct encoding an δAP-3 antisense molecule, siRNA, dominant negative form of δAP-3, anti-δAP-3 antibody, or a small molecule or protein than inhibits complex formation. The method may further comprise administering a second anti-HIV agent to the subject, such as a a nucleoside analog or a reverse transcriptase inhibitor.

In still yet another embodiment, there is provided a pharmaceutical formulation comprising (a) an inhibitor of δ subunit of AP-3 expression and (b) a pharmaceutical carrier, buffer, diluent or excipient.

Embodiments discussed with respect to one embodiment or example of the invention may be employed or implemented with respect to any other embodiment of the invention.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D: Yeast 2-hybrid Analysis of the Gag-AP-3 δ Subunit Interaction. (FIG. 1A) Schematic illustration of AP-3 adapter complex and position of relevant fragments. The original interacting fragment (554-844) isolated from a HeLa cDNA library is shown, together with the position of the AP3D-5′ and AP3D-3′ fragments. (FIG. 1B) Mapping studies of the AP-3δ subunit binding domain within Gag. The interactions between different Gal4 DNA-BD fusion proteins (left graph) and Gal4 AD fusion protein bearing the interacting region of AP-3δ were tested for growth and α-galactosidase activity on solid media (indicated by +) and by liquid β-galactosidase assays (right) in replicate experiments. The top three assays are negative controls. (FIG. 1C) Mapping of the AP-3δ subunit binding domain within MA. MA deletion constructs expressed in yeast were assayed for interaction with the AP3 δ 554-844 fragment as in (FIG. 1B). (FIG. 1D) Fine mapping of the AP-3 δ subunit binding domain within MA.

FIGS. 2A-C: GST pulldown Analysis of the Gag-AP-3 δ Subunit Interaction. (FIG. 2A) HIV-1 MA binds to endogenous AP-3δ subunit. GST fusion proteins representing the indicated regions of Gag bound to glutathione beads were incubated with cell lysates of 293T cells. Immunoblotting for adaptin δ was performed following a series of washes. The input adaptin δ protein from the 293T cell lysate is shown on the right. (FIG. 2B) GST fusion proteins used in GST experiment shown above, analyzed by Coomassie blue staining. (FIG. 2C) The purified interacting fragment (AP-3δ 554-844) was incubated with bead-bound GST-Gag fusion proteins, washed, eluted with SDS-PAGE loading buffer, and eluted proteins analyzed by Coomassie blue staining. The input purified interacting fragment is shown in the rightmost lane, and is indicated in the GST-MA lane by an asterisk.

FIGS. 3A-D: Coimmunoprecipitation of Gag and the AP-3 δ subunit. (FIG. 3A) Coimmunoprecipitation of AP3D-5′ and HIV-1 Gag. 293T cells were cotransfected Gag-myc and either HA-AP3D-5′ or HA-TSG-5′ as a positive control. Proteins were immunoprecipitated using anti-myc antibodies (second and fourth lanes) or using only protein G-sepharose beads (first and third lanes). Coprecipitated HA-AP3D-5′ was detected by immunoblotting with an anti-HA monoclonal antibody (top panel). Cell lysates are shown prior to immunoprecipitation as probed by the anti-HA antibody (middle panel) or anti-myc antibody (bottom panel). (FIG. 3B) Reciprocal coimmunoprecipitation of HIV-1 Gag and AP3D-5′. Cell lysates were prepared as in (FIG. 3A). Proteins were immunoprecipitated from the lysates using anti-HA antibody (second and fourth lanes) or with protein G-sepharose beads alone (first and third lanes). Coprecipitated Gag-myc was detected by immunoblotting with an anti-myc monoclonal antibody. (FIG. 3C) Immunoprecipitation of HIV-1 MA and endogenous AP-3δ subunit. 293T cells were transfected with Gag-myc or SrcΔMAGag-myc expression vectors. Proteins were immunoprecipitated from the untransfected cell lysates (input) or transfected cell lysates using anti-myc polyclonal antisera and detected by Western blot with anti-adaptin δ (top panel). Anti-myc Western blot (middle panel) and anti-adaptin δ Western blot (bottom panel) of the whole cell lysates are shown. (FIG. 3D) Immunoprecipitation of NL4-3 and endogenous AP3 δ subunit. Hela cells were transfected with pNL4-3. Proteins were immunoprecipitated from the untransfected cell laystes (lane 1) and transfected cell lysates (lane 2) using an anti-capsid monoclonal antibody and detected by anti-adaptin δ (top panel) and anti-capsid antibodies (middle panel). Western blot with anti-adaptin δ of the samples prior to immunoprecipitation is shown in the bottom panel.

FIGS. 4A-E: Dominant-negative inhibition of particle assembly/release by AP3D-5′. (FIG. 4A) AP3D-5′ effects on NL4-3 particle assembly. 293T cells were cotransfected with pNL4-3 in a 1:1 mixture with control pcDNA3.1, TSG-5′, or AP3D-5′ expression vectors. p24 antigen release into supernatants was measured at 72 hrs by antigen-capture ELISA. (FIG. 4B) AP3D-5′ effects on Gag/pro-mediated particle assembly. 293T cells were transfected with Gag/pro expression vector in a 1:1 mixture with control pCDNA 3.1, TSG-5′, or AP3D-5′ expression vectors. p24 antigen release into supernatants was measured at 72 hrs by antigen-capture ELISA. (FIG. 4C) Dose-dependent inhibition of assembly by AP3D-5′. 293T cells were cotransfected with NL4-3 expression vector and AP3D-5′ expression vector at the indicated ratios (pNL4-3/AP3D-5′). Supernatant p24 was quantified at 72 hours post-transfection by ELISA. (FIG. 4D) Western blot analysis of dose-dependent inhibition. Cytoplasmic extracts (top panel) and pelleted virus (middle panel) of samples obtained 72 hrs after transfection from the experiment outlined in (FIG. 4C) were harvested and analyzed by Western blot using pooled sera from HIV+ patients. The levels of expressed endogenous AP-3δ subunit (AP-3D) and over-expressed AP3D-5′ as detected by anti-adaptin δ western blot are shown below. (FIG. 4E) Lack of inhibition on particle assembly by AP3D-3′. 293T cells were cotransfected with NL4-3 expression vector and AP3D-3′ expression vector at the indicated ratios, and p24 output in the supernatant was analyzed as in (FIG. 4C).

FIGS. 5A-D: Inhibition of HIV-1 Particle Assembly by siRNA-mediated Depletion of the AP-3 Complex. (FIG. 5A) 293T cells were transfected with a pool of anti-AP-3δ and anti-AP-3 μ siRNA duplexes or with control RNA duplexes. A second transfection with siRNA and pNL4-3 was performed 24 hrs later, and supernatants were harvested 24 hrs following the second transfection (T1). In a parallel experiment, two siRNA transfections were performed 24 hrs apart, followed 24 hrs later by the siRNA/pNL4-3 cotransfection, and harvesting after an additional 24 hrs (T2). Control siRNA transfections were performed separately for each of these experimental arms, and the results are presented as percent of the p24 antigen release as compared with the corresponding control arm. (FIG. 5B) Western blot demonstrating enhanced depletion of AP-3δ and μ subunits at T2 of the same experiment depicted in (FIG. 5A), and the corresponding decrease in released viral particles as indicated by p24. Control Western blot of cell lysates demonstrating AP-1 γ subunit is shown. (FIG. 5C) Depletion of AP-3δ in Hela cells inhibits particle assembly. Cells were transfected with a pool of siRNA directed against AP-3δ or with control RNA duplexes, and 24 hrs later were cotransfected with the same RNA pool and pNL4-3. Top panel demonstrates depletion of AP-3δ subunit. Bottom graph indicates p24 antigen release following control (left) or following specific depletion of the AP-3δ subunit. (FIG. 5D) Resistance of SrcΔMAGag to inhibition by depletion of AP-3. Hela cells were treated with inhibitory RNA or control RNA exactly as in (FIG. 5C) except that SrcΔMAGag was cotransfected instead of pNL4-3. Top panel indicates 6 subunit depletion, bottom graph depicts particle release.

FIGS. 6A-O: AP3D-5′ Alters Intracellular Gag Trafficking. Hela cells were transfected with Gag-CFP and empty control plasmid or with Gag-CFP and YFP-AP3-5′. Cells were fixed at early (12-14 hrs) or late (20 hrs) timepoints post-transfection, permeabilized, immunostained with an anti-CD63 monoclonal antibody, and analyzed for Gag and CD63 distribution using the 63× objective on a Zeiss LSM 510 laser confocal fluorescence microscope. Gag-CFP is shown in green, and CD63 in red. Size bars represent 10 microns. (FIGS. 6A-F) AP3D-5′ inhibits Gag-CD63 colocalization. At early timepoints, Gag is seen predominantly at intracellular sites (FIG. 6A) and colocalizes with CD63 (FIG. 6B and overlay, FIG. 6C). In the presence of YFP-AP3D-5′, even those cells demonstrating punctate Gag-CFP (FIG. 6D) do not demonstrate colocalization with CD63 (FIG. 6E, and overlay, FIG. 6F). (FIG. 6G) Quantitation of Gag/CD63 colocalization. Multiple images of individual cells expressing Gag-CFP and stained for CD63 in the presence or absence of YFP-AP3D-5′ were obtained. The percentage of Gag colocalizing with CD63 was quantified using the MetaMorph software application for 30 evaluable cells in the Gag-CFP alone arm and 25 in the Gag-CFP+YFP-AP3D-5′ arm. (FIG. 6H) YFP image of a pair of cells expressing Gag-CFP and YFP-AP3D-5′. (FIG. 1) Gag-CFP (green)+CD63 (red) image of same pair of cells depicted in (FIG. 6L). (FIG. 6J) YFP image of a pair of cells expressing Gag-CFP and YFP-AP3D-5′. (FIG. 6K) CFP image of the same pair of cells as (FIG. 6N). (FIG. 6L) Plasma membrane pattern of Gag-CFP, 20 hrs post-transfection. (FIG. 6M) Intracellular punctate distribution pattern of Gag-CFP, 20 hrs post-transfection. (FIG. 6N) Intracellular diffuse distribution pattern of Gag-CFP, 20 hrs post-transfection. (FIG. 6O) Quantitation of the three patterns of Gag-CFP distribution at 20 hrs post-transfection for Gag-CFP or Gag-CFP+YFP-AP3D-5′ arms. PM=plasma membrane pattern. Twenty-six cells were counted in the Gag-CFP alone arm, and 32 expressing Gag-CFP with YFP-AP3D-5′.

FIGS. 7A-I: Electron Microscopic Analysis of Particle Assembly Inhibition by TSG-5′ versus AP3D-5′. 293T cells were transfected with NL4-3 DNA alone (FIGS. 7A-C), NL4-3 and TSG-5′ (FIGS. 7D-F), or NL4-3 and AP3D-5′ (FIGS. 7G-I). Cell preparations were fixed, embedded, and sectioned for analysis by transmission electron microscopy on a Philips CM-12 electron microscope. Magnification is 61,600× for FIGS. 7A-F, FIG. 7H and FIG. 7I. Magnification is 17,500× for FIG. 7D and 8800× for FIG. 7G. Scale bars represent 100 nm.

FIGS. 8A-B: Pulse-chase Analysis of Gag in Presence or Absence of AP3D-5′. (FIG. 8A) 293T cells were transfected with a codon-optimized Gag expression construct and pulsed for 20 min with 500 μCi of ³⁵S-cysteine/methionine followed by washing with complete D-MEM containing an excess of cysteine and methionine. Cells were harvested and lysed after a chase of 30 min, 2 hrs, 4 hrs, or 6 hrs. Gag was immunoprecipitated using pooled sera from HIV+ volunteers and analyzed by SDS-PAGE. (FIG. 8B) Phosphorimager analysis of the Pr55^{Gag} bands from the gel represented in the autoradiograph above.

FIG. 9: HIV Infection of HPS2 Fibroblasts. Fibroblasts from HPS2 patients were infected with VSV-G-pseudotyped HIV. The production of HIV particles was monitored by p24 output or by Western blot for released p24. Retroviral transduction was used to replace the defective AP3 subunit (red arrow).

### DESCRIPTION OF ILLUSTRATIVE EMBODIMENTS

I. The Present Invention

The inventors now report here that HIV Gag protein interacts directly with the δ subunit of the AP-3 complex, which interaction is mediated by the amino-terminal α-helical segment of MA. Disruption of this interaction prevents Gag from reaching the MVB compartment and inhibits particle formation. These observations highlight an early step in the HIV assembly pathway that takes Gag to the late endosome/MVB compartment, and suggest that the trafficking of Gag to this compartment is part of the normal productive pathway of HIV-1 assembly.

The data presented here demonstrate that productive HIV particle formation utilizes the AP-3 trafficking pathway. Although the original intracellular membrane to which Gag binds has not been identified, it is suggested that this membrane is either the TGN or a closely-associated tubular sorting endosome, both of which have been identified as intracellular sites of AP-3 localization (Peden et al., 2004; Robinson and Bonifacino, 2001). From this location, the inventors predict that Gag moves to the MVB in a process mediated by AP-3, and subsequently to the plasma membrane. There are several potential implications of this model for HIV particle assembly.

The identification of the δ subunit of AP-3 as a Gag binding partner helps to elucidate unique functions of this subunit. To the inventors' knowledge, there are no known cellular binding partners for the hinge region of this subunit. In yeast, a component of the Vps pathway, Vps41, binds to the carboxyl terminus of the δ subunit homologue Apl5p and contributes to the transport of alkaline phosphatase to the vacuole (Darsow et al., 2001; Rehling et al., 1999). It will be important to identify δ subunit binding partners in mammalian cells in order to better understand the role played by this subunit in vesicular trafficking. It is possible that Gag binds to a site on the δ subunit that is also utilized by cellular cargo proteins, in a manner analogous to that in which Gag competes with the Hrs protein for binding to cellular pools of TSG101.

In summary, Gag binds to the AP-3 complex and uses this route to reach the MVB. This appears to be a productive pathway for HIV particle assembly, even in cell types in which particle assembly takes place predominantly on the plasma membrane. The identification of the Gag-AP-3 interaction elucidates one piece of an increasingly complex pathway by which viral and cellular components interact to generate HIV particles. Moreover, it provides a point of therapeutic intervention in the sceening of anti-HIV agents and the treatment of HIV infection and AIDS. The details of the invention are discussed below.

II. Adaptor Protein (AP) Complexes and Intracellular Trafficking

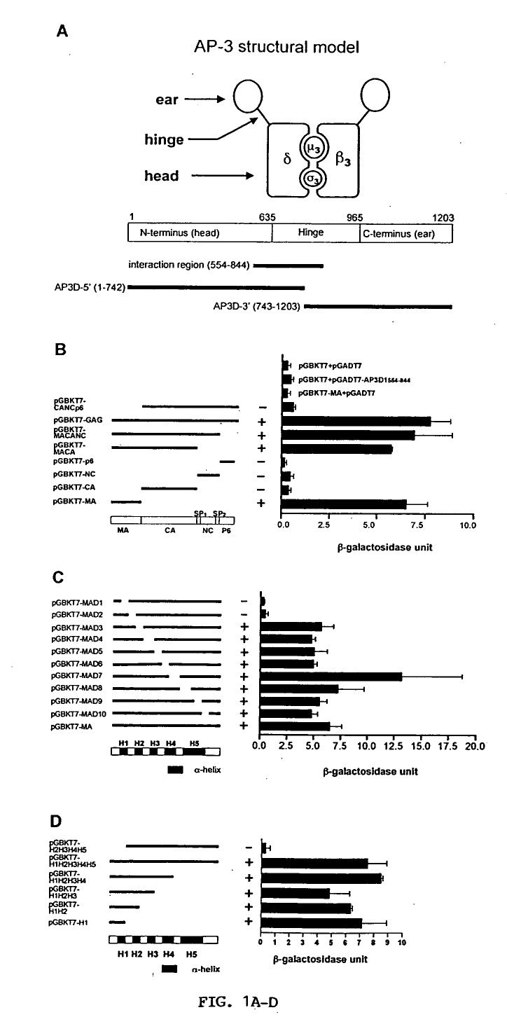

Adaptor protein complexes are heterotetramers that mediate the sorting of cargo proteins to specific membrane compartments within the cell (Boehm and Bonifacino, 2002; Nakatsu and Ohno, 2003; Robinson and Bonifacino, 2001). Four adaptor protein complexes have been described in mammalian cells, designated AP-1 through AP-4. Each adaptor protein complex consists of two large subunits: a β-subunit and a more variable, complex-specific subunit (γ, α, δ, or ε) together with medium-sized (μ) and small (σ) subunits. The relationship between components of the heterotetrameric complex is illustrated in FIGS. 1A-C, in which the carboxy-terminal “ears” of the large subunits are connected to the amino-terminal “head” region through a flexible hinge domain. The μ and σ subunits interact with the head regions of the larger subunits. The μ subunit is responsible for recognition of tyrosine-based sorting signals on the cytosolic tail of membrane glycoproteins, while the σ subunit is important for stabilization of the tetrameric complex (Collins et al., 2002). The AP-1 and AP-2 complexes are important components of clathrin-coated vesicles (Ahle and Ungewickell, 1989; Shih et al., 1995), while the role of clathrin in transport vesicles marked by AP-3 is less clear (Starcevic et al., 2002). The function of AP-2 is perhaps the best defined of the AP complexes, and plays a critical role in receptor-mediated endocytosis (Robinson and Bonifacino, 2001).

AP-3 was first identified through a homology search of cDNA libraries together with database searches (Dell'Angelica et al., 1997; Pevsner et al., 1994; Simpson et al., 1996). Initial immunofluorescence experiments localized AP-3 to the trans-golgi network and to more peripheral endosomal compartments (Dell'Angelica et al., 1997; Simpson et al., 1997). Cells that are deficient in AP-3 demonstrate missorting of lysosomal membrane proteins, including lamp-1, lamp-2, and CD63 (Dell'Angelica et al., 2000; Dell'Angelica et al., 1999; Le Borgne et al., 1998; Rous et al., 2002). Genetic deficiencies of AP-3 subunits in humans lead to disorders collectively termed Hermansky-Pudlak syndrome, in which defects in lysosome-related organelles such as melanosomes and platelet dense granules are apparent (Dell'Angelica et al., 2000; Starcevic et al., 2002). AP-3 has been implicated in the movement of lytic granules to the immunologic synapse (Clark et al., 2003). A recent immuno-electron microscopy study demonstrated that AP-3 localizes to a tubular sorting endosome compartment (Peden et al., 2004).

II. HIV and Gag

A. Viral Overview

HIV-1 is a retrovirus that contains a single-stranded RNA genome of 9 kb. It has 9 genes that encode 15 distinct protein species. The major viral proteins are classified as structural (Gag, Pol, Env), regulatory (Tat and Rev), and accessory (Vpu, Vpr, Vif and Nef). Three major classes of HIV-1 have emerged: M (main), N (new), and O (outlier). There are also various subtypes, called clades. The M group viruses account for >90% of HIV infections worldwide. Clade B is the most common subtype in the Americas and Western Europe, and it differs considerably from those clades found in Asia and Africa. To date, most HIV drug development has targeted clade B.

HIV infection begins with the binding of the virus particle to a host cell. Binding is facilitated by the interaction of the surface env (made up of gp120 and gp41 subunits) protein with its primary receptor CD4. Initial binding to CD4 exposes another portion of the env trimer, which then binds to a coreceptor, usually the chemokine receptor CXCR4 (in the case of T-cell-tropic, or syncytium-inducing strains of HIV) or the chemokine receptor CCR5 (in the case of macrophage-tropic, or nonsyncytium-inducing strains). Coreceptor binding causes the gp41 portion of env to insert into lipid bilayer of the target cell membrane. Next, gp41 fuses the virus and host cell membranes, permitting the viral genetic material and the pol reverse transcriptase to enter the cytoplasm of the host cell. Pol reverse transcribes the viral RNA into DNA, permitting replication.

Preintegration complex, which composed of the cDNA and a number of viral and host proteins, then enters the cell nucleus, where the int enzyme inserts the viral cDNA into the host cell DNA. The resulting integrated “provirus” may remain latent for a little as hours, to as long as years, before becoming transcriptionally active. HIV transcription is complex and controlled by a number of proteins, including both viral (tat) and cellular DNA transcription factors. Transcribed viral RNA is transported out of the nucleus by a number of host and viral factors (e.g., rev). New viral particles are assembled at the plasma membrane and incorporate Gag subunits, Pol, Nef, Env, Vpr, and viral genomic RNA. Following virion assembly, the HIV protease cleaves viral proteins into functional structural and enzymatic components, after which Gag promotes budding of mature virions from the plasma membrane.

Current HIV therapies inhibit the viral replication process at the binding and entry stage (fusion inhibitors), the reverse transcription stage (nucleoside and nonnucleoside reverse transcriptase inhibitors—NRTIs and NNRTIs, respectively), or the protein cleavage stage (protease inhibitors).

B. Gag

The gag gene encodes a 55-kilodalton (kD) precursor protein to the Gag protein. During translation, the N-terminus is myristoylated, thereby triggering its association with cell membranes. Membrane-associated Gag polyprotein sequesters two copies of the viral genome along with other viral and cellular proteins that trigger the budding of the virion. After budding, the polyprotein is cleaved by the viral protease4 (a product of the pol gene), generating four smaller products designated matrix (MA), capsid (CA), nucleocapsid (NC), and p6.

The MA polypeptide is derived from the N-terminus of the Gag precursor, and thus retains the myristoylation site. Most MA molecules are attached to the inner surface of the virion lipid bilayer and are thought to stabilize the particle. A subset of MA is found in deeper layers of the virion as part of the complex which escorts the viral DNA to the nucleus. These MA molecules may be involved in nuclear transport of the preintegration complex in non-dividing cells. The CA protein forms the core of viral particles and interacts with cyclophilin A, which interaction is essential as disruption of this interaction by cyclosporine A inhibits viral replication. The NC protein is responsible for specifically recognizing the packaging signal of HIV, which consists of four stem loop structures located near the 5′ end of the viral RNA. NC binds to the packaging signal through interactions mediated by two zinc-finger motifs. NC also facilitates reverse transcription. p6 mediates interactions between Gag polypeprotein and vpr, leading to incorporation of vpr into assembling virions. The p6 region also contains a so-called “late domain” which is required for the efficient release of budding virions from an infected cell.

III. AP-3δ/Gag Complexes

As discussed above, it is reported here that Gag interacts with δ subunit of AP-3 as part of the pathway by which Gag is trafficked within infected cells and, ultimately, permits viral particle production. A number of previous findings make arguments for the biological relevance of the Gag-AP-3 interaction compelling. It has already been stated that the region of Gag that interacts with AP-3 (MA) has long been implicated in the intracellular trafficking of Gag. Recent observations by multiple groups indicate that Gag trafficks to late endosomes, where Gag and CD63 are found to strongly colocalize (Nguyen et al., 2003; Nydegger et al., 2003; Ono and Freed, 2004; Sherer et al., 2003; von Schwedler et al., 2003). AP-3 is involved in the trafficking of CD63 itself through an interaction between the μ subunit and the lysosomal targeting motif GYEVM on the cytoplasmic tail of CD63 (Dell'Angelica et al., 1999; Rous et al., 2002). Thus, Gag may traffick together with CD63 to late endosomal compartments, explaining the previously recognized high degree of colocalization of these two molecules. Interaction with AP-3 thereby provides a specific means by which Gag can reach the late endosome/MVB, where it may acquire components of the ESCRT pathway that are essential to the normal HIV budding process.

Thus, the inventors propose that the Gag-AP-3 interaction represents an early step in Gag trafficking within the cell, and that in most cells Gag subsequently moves by other mechanisms from the MVB to the plasma membrane. This model of Gag trafficking and particle assembly predicts that essential components of the particle budding machinery may be acquired in the MVB, and this machinery is redirected to the plasma membrane by Gag. The inventors' finding that the rare particles observed budding from the plasma membrane of cells expressing AP3D-5′ exhibited a late defect may reflect the absence of the necessary cellular budding machinery seen when Gag bypasses the MVB. In some cells, such as macrophages, AP-3-mediated trafficking of Gag to the MVB is followed by viral budding into the lumen of the MVB. The intact viral particles within the MVB or “viral exosome” may be transported subsequently to the cell surface for release. It is notable that HIV particles within dendritic cells are transported to contact sites with T cells, a phenomenon known as the “virologic synapse” (McDonald et al., 2003). AP-3 has been shown to play a role in the movement of lytic granules to the immunologic synapse (Clark et al., 2003). It will thus be important in the future to determine if AP-3 is involved in the movement of particle-laden MVBs to the virologic synapse in a manner analogous to its role in directing lytic granules to the immunologic synapse.

IV. Screening

The present invention further comprises methods for identifying inhibitors of Gag-AP-3 interactions. These assays may be performed in cells or in cell-free environments, and may comprise random screening of large libraries of candidate substances. Alternatively, the assays may be used to focus on particular classes of compounds selected with an eye towards structural attributes that are believed to make them more likely to inhibit the formation of Gag-AP-3 complexes.

To identify an inhibitor in accordance with the present invention, one generally will determine the ability of candidate compound to inhibit Gag-AP-3 complex formation. For example, a method generally comprises:
(a) providing δ subunit of adaptor protein-3 (δAP-3) and Gag under conditions permitting the formation of an δAP-3/Gag complex;
(b) contacting said first substance with δAP-3 and Gag; and
(c) assessing the formation of δAP-3/Gag complex,
   wherein a decrease in complex formation, as compared to complex formation in the absence of the first substance, identifies the candidate substance as an inhibitor of δAP-3/Gag complex formation, as well as a potential inhibitor of HIV and AIDS. Such assays can be performed in cell free environments, but also may be conducted in isolated cells, organs, or in living organisms.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may potentially inhibit the formation of δAP-3/Gag complexes. The candidate substance may be a protein or fragment thereof, a small molecule, or even a nucleic acid. It may prove to be the case that the most useful pharmacological compounds will be compounds that are discovered through high-throughput screens of large compound libraries. Using lead compounds to help develop improved compounds is known as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration, or which may affect the function of various other molecules. In one approach, one would generate a three-dimensional structure for a target molecule such as Gag or δAP-3, or a fragment thereof. This could be accomplished by x-ray crystallography, computer modeling, or by a combination of both approaches.

It also is possible to use antibodies to ascertain the structure of a target compound, activator, or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecular libraries that are believed to meet the basic criteria for useful drugs in an effort to “brute force” the identification of useful compounds. Screening of such libraries, including combinatorially-generated libraries (e.g., peptide libraries), is a rapid and efficient way to screen large number of related (and unrelated) compounds for activity. Combinatorial approaches also lend themselves to rapid evolution of potential drugs by the creation of second, third, and fourth generation compounds modeled on active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for AP-3. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

A technique for high throughput screening of compounds is described in WO 84/03564. Large numbers of small peptide test compounds are synthesized on a solid substrate, such as plastic pins or some other surface. Such peptides could be rapidly screening for their ability to inhibit δAP-3/Gag complexes, or to bind regions on δAP-3 or Gag.

C. In Cyto Assays

The present invention also contemplates the screening of compounds for their ability to inhibit δAP-3/Gag complex formation in cells. Various cell lines can be utilized for such screening assays, including cells specifically engineered for this purpose. In addition, genetic constructs for use in transforming cells for such assays are described elsewhere in this document.

D. In Vivo Assays

In vivo assays involve the use of various animal models of HIV infection. Due to their size, ease of handling, and information on their physiology and genetic make-up, mice are a preferred embodiment, especially for transgenics. However, other animals may provide better models of HIV infection, such as monkeys (including chimps, gibbons and baboons). Treatment of animals with test compounds will involve the administration of the compound, in an appropriate form, to the animal. Administration will be by any route that could be utilized for clinical purposes. Determining the effectiveness of a compound in vivo may involve a variety of different criteria. Also, measuring toxicity and dose response can be performed in animals in a more meaningful fashion than in in vitro or in cyto assays.

E. Specific Assay Formats

The present invention may utilize a variety of specific assay formats. For example, the ability of δAP-3 and Gag to form a complex may be assessed using a two-hybrid system. Yeast two-hybrid assays are described in U.S. Pat. No. 5,283,173 (incorporated herein by reference), and are techniques well known to those of skill in the art. Briefly, the method is designed to detect an interaction between a first test protein and a second test protein, in vivo, using reconstitution of the activity of a transcriptional activator. Two chimeric proteins that express hybrid proteins are prepared. The first hybrid protein contains the DNA-binding domain of a transcriptional activator fused to the first test protein, while the second hybrid protein contains a transcriptional activation domain fused to the second test protein. If the two test proteins interact, the two domains of the transcriptional activator are brought into close proximity, resulting in the transcription of a marker gene that contains a binding site for the DNA-binding domain. An assay can be performed to detect activity of the marker gene.

All yeast two-hybrid systems share a set of common elements: 1) a plasmid that directs the synthesis of a “bait”; the bait is a known protein which is fused to a DNA binding domain, 2) one or more reporter genes (“reporters”) with upstream DNA binding sites for the bait, and 3) a plasmid that directs the synthesis of proteins fused to activation domains and other useful moieties (“activation tagged proteins” or “prey”). All current systems direct the synthesis of proteins that carry the activation domain at the amino terminus of the fusion, facilitating the expression of open reading frames encoded by cDNAs. DNA binding domains used in the yeast two-hybrid systems include the native E. coli LexA repressor protein (Gyuris et al., 1993), and the GAL4 protein (Chien et al., 1991). Some reporter genes that may be utilized in the yeast system included HIS3, LEU2, and lacZ.

Although most two-hybrid systems use yeast, mammalian variants may also be utilized. In one system, interaction of activation tagged VP16 derivatives with a Gal4-derived bait drives expression of reporters that direct the synthesis of Hygromycin B phosphotransferase, Chloramphenicol acetyltransferase, or CD4 cell surface antigen (Fearon et al., 1992). In another system, interaction of VP16-tagged derivatives with Gal4-derived baits drives the synthesis of SV40 T antigen, which in turn promotes the replication of the prey plasmid, because the plasmid carries a SV40 origin (Vasavada et al., 1991).

Protein-protein interactions may also be studied by using biochemical and immunologic techniques, such as fluoresence energy transfer where different molecules are both labeled with appropriate fluorescent donor-acceptor pairs, co-immunoprecipitation, double-determinant Western blot and ELISAs (e.g., sandwich ELISA), which are well known to those skilled in the art. In order to render the complexes more stable for examination, cross-linking may be performed prior to testing. One may also perform band shift or differential chromatography assays that assess complex formation by speed of migration in electrophoretic media (e.g., gels) or chomatrographic substrates (paper, columns, etc.).

V. Treating HIV Infection

The present invention also provides for the treatment of HIV infections and acquired immunodeficiency disease (AIDS). By use of the term “treatment”, applicants envision that the effects upon the patient may include one or more of reduction in viral burden, increase in T cell count, increased survival, increased exercise capacity, decreased dependency on traditional drug therapy, and reduced hospital stays.

The following constitute examples of various agents that may prove useful in inhibiting δAP-3/Gag complex formation, and thus in inhibiting HIV infection and AIDS.

A. Pharmaceutical Inhibitors

Inhibiting AP-3 as a pharmaceutical treatment has not been reported. Nonetheless, one could easily screen large compound libraries using the screens described herein to discover small molecules capable of inhibiting AP-3 expression or function. Furthermore, standard medicial chemistry approaches could be applied to these compounds to enhance or modify their activity so as to yield novel compounds.

B. Antisense Constructs

An alternative approach to inhibiting AP-3 would be utilization of antisense technology. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit AP-3 gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

C. Ribozymes

Another general class of inhibitors would be RNA-specific ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). It has also been shown that ribozymes can elicit genetic changes in some cells lines to which they were applied; the altered genes included the oncogenes H-ras, c-fos and genes of HIV. Most of this work involved the modification of a target mRNA, based on a specific mutant codon that was cleaved by a specific ribozyme.

D. RNAi (siRNA)

RNA interference (also referred to as “RNA-mediated interference” or RNAi) is another mechanism by which AP-3 expression could be modulated in a way similar to that of the antisense methodology. One can envision instances when AP-3 RNAs could be reduced or eliminated, leading to decreased expression of AP-3. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi (or siRNA) offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single-stranded oligomers into a double-stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et al. (2001) wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single-strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Treatment regimens would vary depending on the clinical situation. However, long term maintenance would appear to be appropriate in most circumstances. It also may be desirable treat hypertropby with agonists of MCIP-1-38 intermittently, such as within brief window during disease progression.

E. Antibodies

In certain aspects of the invention, antibodies may find use as antagonists of AP-3 activity or expression. As used herein, the term “antibody” is intended to refer broadly to any appropriate immunologic binding agent such as IgG, IgM, IgA, IgD and IgE. Generally, IgG and/or IgM are preferred because they are the most common antibodies in the physiological situation and because they are most easily made in a laboratory setting.

The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

Single-chain antibodies are described in U.S. Pat. Nos. 4,946,778 and 5,888,773, each of which are hereby incorporated by reference.

“Humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

F. Peptide Aptamers

Peptide aptamers represent yet another potential mechanism for either inhibiting proper AP-3 folding or disturbing/treating a secondary interaction with Gag. Recently, the ability to manipulate individual genes has driven the development of reverse genetics, in which the function of genes is inferred from the phenotypes that arise from their mutation. In diploids, reverse genetics also typically requires generation of homozygotes in the mutated gene. To circumvent this requirement, a number of dominant “reverse genetic” methods to inactivate gene function have been devised, including inhibition by drugs, expression of dominant-negative proteins, injection of antibodies, expression of antisense RNAs, expression of nucleic acid aptamers, and expression of peptide aptamers (Geyer et al., 1999).

The ability to specifically interfere with the function of proteins of pathological significance has been a goal for molecular medicine for many years. Peptide aptamers are proteins that contain a conformationally constrained peptide region of variable sequence displayed from a scaffold (Geyer et al., 1999). Peptide aptamers comprise a new class of molecules, with a peptide moiety of randomized sequence, which are selected for their ability to bind to a given target protein under intracellular conditions (Hoppe-Seyler et al., 2004). They have the potential to inhibit the biochemical activities of a target protein, can delineate the interactions of the target protein in regulatory networks, and identify novel therapeutic targets. Peptide aptamers represent a new basis for drug design and protein therapy, with implications for basic and applied research, for a broad variety of different types of diseases (Hoppe-Seyler et al., 2004).

Peptide aptamers from combinatorial libraries can be dominant inhibitors of gene function. Researchers have used two-hybrid systems to select aptamers based on Escherichia coli thioredoxin (TrxA) that recognize specific proteins and allelic variants. Apterms have been selected against Cdk2 (Colas et al., 1996), Ras (Xu et al, 1997), E2F (Fabbrizio et al., 1999), and HIV-1 Rev (Cohen et al., 1998). Apterms have been used in mammalian cells (Cohen et al, 1998) and in Drosophila melanogaster (Kolonin et al., 1998). These recent results demonstrate the power and potential utility of peptide aptamers, both as stand-alone therapeutics and even as a potential class of inhibitors of nuclear export. As such they could be used to block nuclear export, or they could be used in conjunction with an inhibitor of nuclear export as a dual or combination therapy.

G. Combined Therapy

In addition to use as a mono-therapy, inhibitors of δAP-3/Gag complexes may also improve the efficacy of other traditional anti-viral compounds as well. In particular, the use of NRTIs such as didexoyinosine, dideoxycytidine and azidothymidine in combination therapies are envisioned. Other combination therapies are envisioned, such as non-nucleotide reverse transcriptase inhibitors, integrase inhibitors, protease inhibitors, or inhibitors of virus entry, such as T-20. Such a strategy is important not only in the creation of more effective therapies, but in reducing the chance that drug-resistant viruses will develop.

To inhibit virus replication and thereby limit infection and T cell loss, using the methods and compositions of the present invention, one will treat a patient with a δAP-3/Gag complex inhibitor composition and a traditional antiviral therapeutic. This process may involve administration of both therapies at the same time, for example, by administration of a single composition or pharmacological formulation that includes both agents, or by administering to said patient two distinct compositions or formulations, at the same time.

Alternatively, the traditional therapy may precede or follow the present Gallium composition treatment by intervals ranging from minutes to weeks. It is also conceivable that more than one administration of either treatment will be desired. Various combinations may be employed, where the δAP-3/Gag inhibitor composition is “A” and the traditional therapeutic is “B”:

A/B/A B/A/B B/B/A A/A/B A/B/B B/A/A B/B/B/A
B/B/A/B A/A/B/B A/B/A/B A/B/B/A B/B/A/A B/A/B/A
B/A/A/B A/A/A/B B/A/A/A A/B/A/A A/A/B/A A/B/B/B
B/A/B/B

H. Administration of Adjunct Therapeutic Agents

Pharmacological therapeutic agents and methods of administration, dosages, etc., are well known to those of skill in the art (see for example, the “Physicians Desk Reference,” Goodman & Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such invidual determinations are within the skill of those of ordinary skill in the art.

I. Drug Formulations and Routes for Administration to Patients

It will be understood that in the discussion of formulations and methods of treatment, including references to any compounds, are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, or used as a suppository, or be formulated for subcutaneous, intra-arterial, intravenous, intramuscular, intraperitoneal, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds may also be administered in the form of suppositories for rectal administration of the drug. These compositions can be prepared by mixing a therapeutic agent with a suitable non-irritating excipient which is solid at ordinary temperatures, but liquid at the rectal temperature and will therefore melt in the rectum to release the drug. Such materials are cocoa butter and polyethylene glycols.

For topical use, creams, ointments, jellies, gels, epidermal solutions or suspensions, etc., containing a therapeutic compound are employed. For purposes of this application, topical application shall include mouthwashes and gargles.

Formulations may also be administered as nanoparticles, liposomes, granules, inhalants, nasal solutions, or intravenous admixtures

The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

VI. Vectors for Cloning, Gene Transfer and Expression

Within certain embodiments, expression vectors are employed to express various products including Gag, AP-3 or the δ subunit thereof, antisense molecules, ribozymes, single-chain antibodies or interfering RNAs. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In some embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 1 and 2 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 1 and Table 2). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 1
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa et |
| | | al., 1987, Karin et al., |
| | | 1987; Angel et al., 1987b; |
| | | McNeall et al., 1989 |
| MMTV (mouse | Glucocorticoids | Huang et al., 1981; Lee et |
| mammary | | al., 1981; Majors et al., |
| tumor virus) | | 1983; Chandler et al., |
| | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macro globulin | IL-6 | Kunz et al., 1989 |
| Vimentin | Serum | Rittling et al., 1989 |
| MHC Class I | Interferon | Blanar et al., 1989 |
| Gene H-2κb | | |
| HSP70 | E1A, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

B. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

D. Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, Gag, pol, and env that code for capsid proteins, polymerase enzyme, and envelope components, respectively. A sequence found upstream from the Gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the Gag, Pol, and Env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al, 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the Gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, E. P. App. 273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAAs (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

VII. Preparing Antibodies to AP-3 or Gag

In yet another aspect, the present invention contemplates the use of antibodies that may bind to AP-3 or the δ subunit thereof, or to Gag. An antibody can be a polyclonal or a monoclonal antibody, it can be humanized, single chain, or even an Fab fragment. In a preferred embodiment, an antibody is a monoclonal antibody. Means for preparing and characterizing antibodies are well known in the art (see Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to AP-3 or Gag antigen epitopes.

In general, both polyclonal, monoclonal, and single-chain antibodies against Ku may be used in a variety of embodiments. A particularly useful application of such antibodies is in purifying native or recombinant AP-3 or Gag, for example, using an antibody affinity column. The operation of all accepted immunological techniques will be known to those of skill in the art in light of the present disclosure.

Means for preparing and characterizing antibodies are well known in the art (see, e.g., Harlow and Lane, 1988; incorporated herein by reference). More specific examples of monoclonal antibody preparation are given in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified PKD protein, polypeptide or peptide or cell expressing high levels of PKD. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

VIII. Examples

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

Plasmid Constructs. The pNL4-3 expression plasmid was obtained from Dr. Malcolm Martin through the NIH AIDS Reference and Reagent Program. Expression constructs for the AP3 δ subunit (AP3D) were derived from a full-length cDNA construct kindly provided by Margaret Robinson (University of Cambridge, UK). PCR cloning methods were used to generate expression constructs for AP3 δ subunit residues 1-742 (AP3D-5′) and AP3 δ subunit residues 743-1203 (AP3D-3′) in the mammalian expression vector pcDNA3.1 (Stratagene) using the EcoR1 and Xho1 sites. An HA-tag was introduced into the N-terminus of AP3D-5′ by PCR cloning; this construct was placed into the BamH1 and Xho1 sites of pCDNA 3.1. DNA encoding AP3 δ subunit residues 1-742 was also amplified and inserted into pEYFP-Cl (Clontech) to generate YFP-AP3D-5′. A full-length TSG101 gene was cloned and sequenced from the HeLa cDNA library, and a fragment representing codons 10-240 was amplified and cloned into EcoR1 and Xho1 sites of pCMV-HA (Clontech). This vector construction placed an HA tag at the N-terminus of the TSG-5′ fragment previously reported to produce a late budding defect (Demirov et al., 2002). Codon-optimized Gag was utilized in some experiments, and was derived from the pVRC3900 expression plasmid provided by Gary Nabel (VRC, NIH) (Huang et al., 2001). An expression plasmid for codon-optimized Gag with a carboxyl-terminal myc epitope tag was generated by PCR cloning into the HindIII and EcoR1 sites of pCDNA3.1. Two constructs were generated that express codon-optimized Gag with a deletion of the entire matrix coding region (codons 1-132) and fused to the N-terminal 9 residues of v-src. One of these constructs was generated with a carboxyl-terminal myc epitope tag (SrcΔMAGag-myc) using HindIII and EcoR1 sites, while the other included the stop codon at the end of the gag gene (SrcΔMAGag) cloned into HindIII and BamH1 sites of pcCNA3.1. Matrix deletion constructs (pMAD1-10) were provided by Max Essex (Harvard University, Boston, Mass.) (Yu et al., 1992). The matrix coding regions of pMAD1-10 were cloned using PCR amplification into yeast 2-hybrid vector pGBKT7 for yeast expression (Clontech) using the Nde1 and BamH1 sites. A series of Gag protein truncation products were also cloned into pGBKT7 using these sites for mapping of the interacting domains in directed yeast 2-hybrid assays. GST fusion constructs with specific regions derived from the codon-optimized gag gene were created (MA, CA, NC, p6, MACA, MACANC, CANCp6, full-length Gag) by PCR cloning into the BamH1 and EcoR1 sites of pGEX-2T (Amersham Pharmacia), as was a GST fusion with the AP-3 binding domain (residues 554-844). All expression constructs were verified by sequencing throughout the amplified regions. Expression vector Gag-CFP has been previously described (Derdowski et al., 2004) and the construct 3-CCCC for Gag-Pol expression was a gift from Hans-Georg Krausslich (University of Hamburg, Germany) (Wodrich et al., 2000).

Yeast Two-Hybrid Assays. Two-hybrid library screening was performed using full-length codon-optimized Gag (codons 1-1503) as bait fused to codons 1-147 of the Gal4 DNA binding domain (BD) in vector pGBKT7 (Clontech). Prey constructs were derived from a human Hela cDNA library fused to the C-terminus of codons 768-881 from the Gal4 activation domain in vector pACT2 (Clontech). Yeast strain AH109, containing four reporter genes (ADE2, HIS3, lacZ, and MEL1) was co-transformed with bait and prey constructs according to the manufacturer's instructions (Matchmaker two-hybrid system, Clontech). Candidate positive prey constructs were selected by growth on selective media and by production of color by α-galactosidase secretion. Candidate prey constructs were isolated from yeast colonies and cotransformed into naïve yeast cells to confirm a specific interaction with the Gag-Gal4 BD construct. The identity of the isolated prey DNA was confirmed by automated sequencing and comparison with the database. Directed two-hybrid interaction experiments were performed by cotransformation of recombinant pGBKT7 and pGADT7 constructs into yeast strain AH109. The cotranformants were plated on quadruple selection medium (-Leu/-Trp/-Ade/-His) with X-α-gal. Liquid β-galactosidase assays were performed using CPRG as substrate. For liquid culture assays, cells in mid-log phase were processed as described (Schneider et al., 1996) and β-galactosidase units were quantitated in a spectrophotometer according to a protocol from the manufacturer (Matchmaker two-hybrid system, Clontech).

Protein Expression and GST Pulldown. GST fusion proteins were expressed in E. coli BL21 (DE3) cells (Novagen) following 0.1 mM IPTG induction, and were subsequently purified using glutathione-sepharose beads. The immobilized GST fusion proteins were incubated for 2-4 hrs at 4° C. with 293T cell lysates, washed extensively in RIPA buffer (PBS with 1% NP-40, 0.1% SDS), and eluted with SDS-PAGE loading buffer followed by detection by Western blotting using the monoclonal antibodies indicated below. The interaction region fragment was produced similarly as a GST fusion and then exposed to ρ-amino-benzamidine-agarose beads for removal of thrombin. The interaction domain protein was then dialyzed against PBS in a Slide-A-Lyzer Dialysis Cassette (Pierce). For interaction experiments, 30 μl of a concentrated preparation was added to each of the Gag-GST fusion proteins, and an equal amount analyzed on the gel used to detect the eluted proteins by Coumassie staining.

Antibodies and coimmunoprecipitation. 293T cells were maintained in Dulbecco's modified Eagle medium with 10% fetal bovine serum and antibiotics at 37° C. in 5% CO₂ and grown in 10 cm² culture dishes. Transfections were performed by the calcium phosphate method. Cells were harvested 36-48 hr post-transfection, washed in phosphate-buffer saline, exposed to hypotonic buffer (10 mM Tris pH 8.0, 1 mM EDTA) with protease inhibitors for 15-20 min on ice, broken by Dounce homogenization, and the lysate solution adjusted to 0.15M NaCl, 1 mM MgCl₂. The cell lysates were centrifuged at 1500×g for 10 min, and the supernatants were immunoprecipitated with anti-HA, anti-Myc, or anti-24 monoclonal antibody 183-H12-5C (provided by Bruce Chesebro and Kathy Wehrly through the NIH AIDS Research and Reference Reagent Program) for 2 hr at 4° C., washed extensively in wash buffer (50 mM Tris pH 7.4, 150 mM NaCl, 5 mM EDTA, 0.5% NP-40, 0.02% sodium azide) and detected by Western blotting with the indicated antibodies. The detection of Gag-AP-3 interactions in cells expressing the NL4-3 virus was performed in Hela cells. In experiments in which the protein content of viral particles were directly analyzed by Western blotting, they were first pelleted through 20% sucrose in a Beckman SW 28 rotor at 28,000 rpm at 4° C. Antibodies used for detection of AP subunits were from BD Biosciences catalog #611328 for AP-3 δ subunit, catalog #610900 for AP-3 μ subunit, and catalog #610385 for AP-1 γ subunit. In some experiments heat-inactivated sera pooled from ten HIV-positive donors was used.

RNA interference (RNAi). Twenty-one nucleotide siRNA duplexes with symmetric two nucleotide 3′-UU overhangs were obtained from Dharmacon, and correspond to the following AP-3 δ subunit targets: nucleotides 176-194 (TCTGCAAGCTGACGTATTT (SEQ ID NO:1)), 489-507 (GAAGAAGGCTGTGCTGATC (SEQ ID NO:2)), 2438-2456 (GCGAGAAACTGCCTATTCA (SEQ ID NO:3)), and 2493-2511 (GAAGGACGTTCCCATGGTA (SEQ ID NO:4)). An additional siRNA reagent was generated in the inventors' laboratory using the Silencer siRNA construction kit (Ambion) following the manufacturer's instructions and targeting the AP-3 μ subunit nucleotides 237-255 (TTGAGTTCCTACATCGAGT (SEQ ID NO:5)). Control siRNA duplexes were duplex I sense strand (GCUGAGUAUACGCGGAUGUUU (SEQ ID NO:6)) with 53% GC content, and control duplex II (ACCACCAACAUAUCUACGCUU (SEQ ID NO:7)), with 47% GC content. siRNA transfection was performed with Lipofectamine 2000 (Invitrogen). For each siRNA knockdown experiment, 100 nM siRNA was used in experimental and control arms, and the control siRNA used was matched for GC content to the active arm.

Immunofluorescence microscopy. Hela cells maintained in D-MEM/10% FBS medium were grown on glass cover slips in 6-well plates and were transfected using with Lipofectamine 2000 (Invitrogen). Transfected cells were fixed with 3.8% formaldehyde in sodium phosphate buffer, stabilized in cytoskeletal stabilization buffer (100 mM PIPES, 1 mM EDTA, 3% PEG6000, pH6.9), permeabilized with 100 mM PIPES/1 mM EDTA/3% PEG6000/0.1% Triton X-100, and blocked with 5% bovine serum albumin in phosphate-buffered saline. Cells were incubated with mouse anti-Lamp-3 (1:100, Santa Cruz Biotechnology Inc.), washed with PBS, incubated with goat anti-mouse Alexa 546-cojugated antibodies (1:3000, Molecular Probes). Confocal images were acquired using a Zeiss LSM510 laser-scanning confocal microscope (Carl Zeiss Inc.), and data analysis performed with MetaMorph software (Universal Imaging Corporation).

Electron Microscopy. Transfected 293T cells were pelleted in 1.5-ml microcentrifuge tubes at 2,000×g for 10 min. The pelleted cells were fixed, sectioned and stained as previously described (Sandefur et al., 2000). Images were obtained on a Philips CM-12 electron microscope equipped with a high-resolution CCD camera.

### EXAMPLE 2

### Results

HIV-1 Gag Binds Specifically to the δ Subunit of the AP-3 Complex. The inventors performed a yeast two-hybrid screen of a HeLa cDNA library using full-length HIV-1 Gag as bait in order to identify novel Gag binding partners. This screen identified TSG101 and cyclophilin B, two previously identified Gag-interacting proteins (Franke et al., 1994; Garrus et al., 2001; VerPlank et al., 2001), as well as several new candidate Gag binding partners. One new candidate chosen for further evaluation was the δ subunit of AP-3. This screen identified a fragment representing amino acids 554-844 of the δ subunit, which overlaps the hinge region and the C-terminal portion of the body of AP-3 δ. In subsequent experiments, the inventors confirmed this interaction in yeast by measuring β-galactosidase levels in liquid culture assays (FIG. 1B). Additional truncation constructs narrowed the Gag binding region on AP-3 δ to amino acids 663-742, which are located entirely within the hinge region (data not shown).

Directed yeast two-hybrid experiments were used to map the AP-3 δ subunit binding site within Gag. Deletion of MA from Gag eliminated specific binding, while C-terminal truncations of Gag that retained an intact MA region reproduced the binding characteristics of the full-length molecule (FIG. 1B). Next, the inventors examined individual fragments representing each major cleavage product of Gag (MA, CA, NC, p6); only the MA fragment bound at levels significantly above background (FIG. 1B). The region within MA responsible for the observed binding was then investigated using a series of deletion constructs. A set of previously-described nested deletions within MA were evaluated (Yu et al., 1992), and revealed loss of interaction with deletions surrounding the N-terminal α-helix of MA (MAD-1 and MAD-2, FIG. 1C). To confirm the importance of the N-terminal α helix (H1), a fragment expressing the N-terminus of MA up to residue 19 demonstrated interaction in yeast (pGBKT7-H1), while a specific deletion of this same fragment failed to bind to the AP-3 delta subunit (FIG. 1D). A N-terminal fragment of MA truncated after residue 12 failed to interact (data not shown). The inventors conclude from these studies that the N-terminal α helix of MA, a region previously implicated in Gag trafficking (Ono et al., 2000), is the binding site for the AP-3 δ subunit.

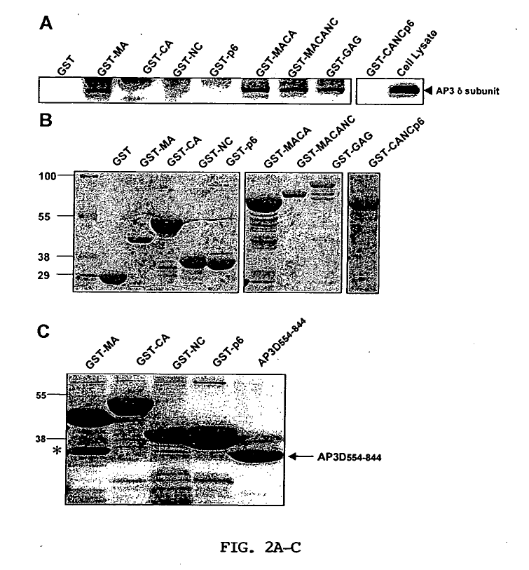

Next, the inventors sought to verify the Gag-AP-3 delta interaction using recombinant Gag protein produced in E. coli. A series of Gag-GST fusion constructs representing major subdomains of Gag were purified on glutathione-agarose beads. Cell lysates from 293T cells were prepared, and the ability of Gag to bind the cellular AP-3 δ subunit was evaluated. No interaction was seen with GST alone or with GST fusions with CA, NC, or p6 (FIG. 2A). Cellular AP-3 delta bound to full-length GST-Gag, Gag lacking p6 (GST-MACANC), Gag lacking NC and p6 (GST-MACA), and with GST-MA alone (FIG. 2A). To further analyze this interaction, the inventors examined the ability of a purified protein fragment representing the interacting domain of the AP-3 delta subunit (amino acids 554-844) to bind directly to a panel of GST-Gag fusion proteins. The purified fragment bound to GST-MA and not to fusion proteins representing other major regions of Gag (FIG. 2C, asterisk). These results indicate that there is a direct and specific protein-protein interaction between the MA region of Gag and the AP-3 δ subunit.

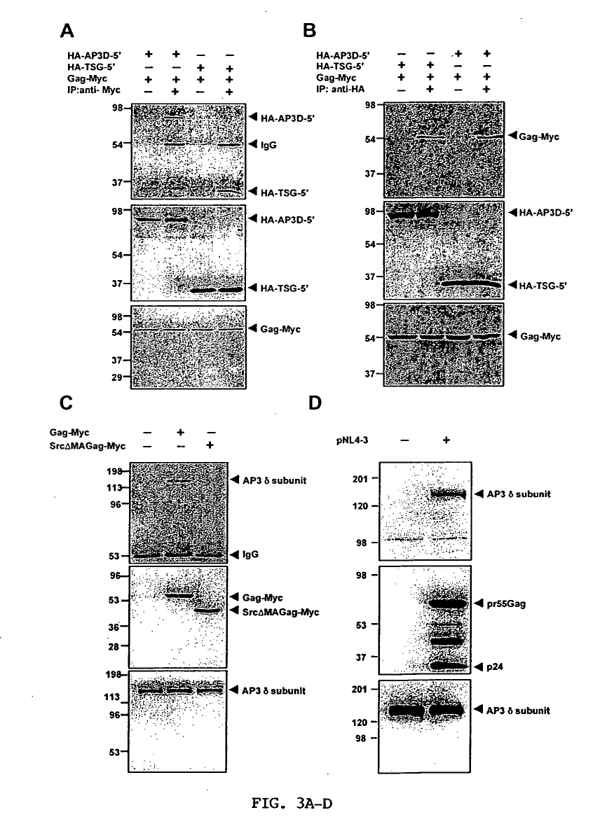

Gag Interacts with the AP-3 δ Subunit in Mammalian Cells. An N-terminal fragment of the AP-3 δ subunit (amino acids 1-742) that includes the Gag binding domain was generated with an N-terminal HA epitope tag. The HA-tagged AP-3 fragment, termed HA-AP3D-5′, was expressed together with a full-length Gag molecule bearing a C-terminal myc epitope tag (Gag-myc) in 293T cells. Immunoprecipitation of Gag demonstrated interaction with HA-AP3D-5′ as detected by anti-HA immunoblotting (FIG. 3A). HA-tagged TSG-5′, an N-terminal fragment of TSG101 that includes the p6 binding region, was coprecipitated with Gag-myc in this assay as a positive control. The reciprocal immunoprecipitation was then performed (immunoprecipitation with anti-HA followed by anti-myc immunoblotting), and again demonstrated the co-immunoprecipitation of Gag-myc with HA-AP3D-5′ (FIG. 3B).

In order to detect the interaction of Gag with endogenous AP-3 in mammalian cells, immunoprecipitation of Gag-myc was next performed using an anti-myc monoclonal antibody in the absence of overexpression of the AP-3 δ subunit, followed by immunoblotting for detection of the endogenous AP-3 δ subunit. Gag-myc precipitated the full-length endogenous AP-3 δ subunit (FIG. 3C). A particle-competent Gag-myc construct bearing the v-src myristylation signal and lacking MA failed to precipitate the AP-3 δ subunit, confirming the previous mapping data (SrcΔMAGag-myc, lane 3 of FIG. 3C). To further examine this interaction in mammalian cells, a full-length proviral construct (pNL4-3) was introduced into Hela cells by transfection. Wild-type Gag was immunoprecipitated using an anti-CA monoclonal antibody, and the endogenous AP-3 δ subunit was readily detected by immunoblotting (FIG. 3D). Taken together, these results demonstrate that Gag interacts with the AP-3 δ subunit in mammalian cells when Gag is expressed in the absence of other viral gene products and in the context of expression of an infectious provirus.

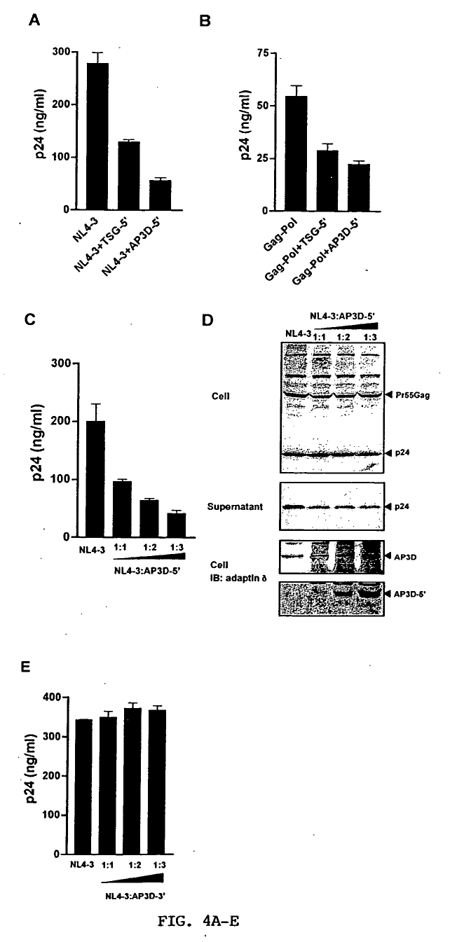

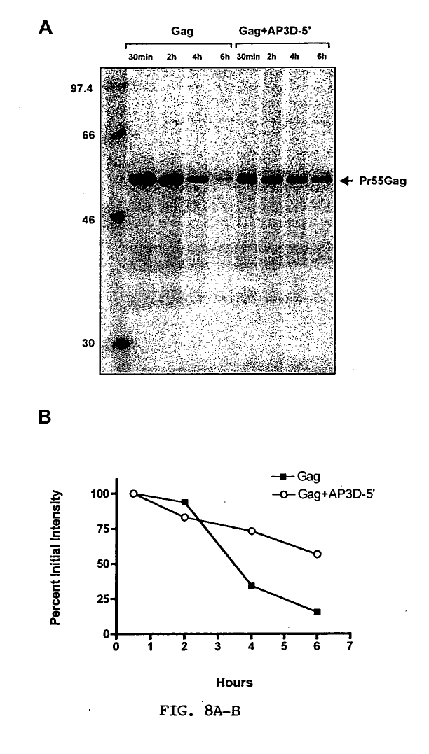

An N-terminal fragment of the AP-3 delta subunit acts as a dominant-negative inhibitor of particle assembly. Next, the inventors performed experiments designed to investigate the biological significance of the Gag-AP-3 interaction. They reasoned that expression of a fragment of AP-3 δ overlapping the Gag binding domain could block the interaction of Gag with the endogenous AP-3 complex. To study the effects of this intervention on particle formation, the inventors expressed AP3D-5′ together with pNL4-3 in 293T cells, and measured particle release by p24 antigen ELISA. AP3D-5′ expression inhibited particle assembly/release from 293T cells in a manner similar to that of a dominant-negative TSG101 construct (FIG. 4A). In repeated experiments, the quantitative inhibitory effect of the AP3D-5′ construct transfected at a 1:1 ratio with pNL4-3 was slightly greater than that of TSG-5′ (FIGS. 4A-B, and data not shown). The effect of AP3D-5′ and TSG-5′ on particle release following expression of HIV gag and protease genes in 293T cells was next determined. AP3D-5′ demonstrated a significant inhibition of particle release in this context, eliminating the possibility that Env or additional viral gene products are required for AP3D-5′-mediated inhibition of assembly (FIG. 4B). The inhibitory effect of AP3D-5′ was dose-dependent, as demonstrated by increasing inhibition of p24 release from NL4-3-expressing cells (FIG. 4C). Western blotting of cell lysates and of pelleted virion particles revealed an AP3D-5′ dose-dependent decrease in released p24 antigen that corresponded well with the ELISA results (FIG. 4D). The inventors considered the possibility that the inhibition of particle release seen upon over-expression of AP3D-5′ was due to cellular toxicity or enhanced degradation of Pr55^{Gag}. However, intracellular levels of Pr55^{Gag} and of endogenous AP-3 delta subunit did not decrease substantially at ratios of pNL4-3 DNA/AP3D-5′ DNA ranging from 1:1 to 1:3 (FIG. 4D). To more directly address this possibility, the inventors performed pulse-chase analysis of Gag in cells transfected with AP3D-5′ DNA or with control DNA. The half-life of Pr55^{Gag} in cells expressing AP3D-5′ was prolonged compared to that observed in cells lacking AP3D-5′, reflecting the fact that Pr55^{Gag} was retained within cells in which the dominant-negative construct was expressed (FIGS. 8A-B). The inhibitory effects of AP3D-5′ on particle release were not seen upon expression of a C-terminal fragment of AP-3 δ subunit that lacked the Gag interaction domain (AP3D-3′, FIG. 4E). Taken together, these data demonstrate that AP3D-5′ acts as a dominant-negative inhibitor of assembly through a mechanism that does not induce enhanced degradation of Gag.

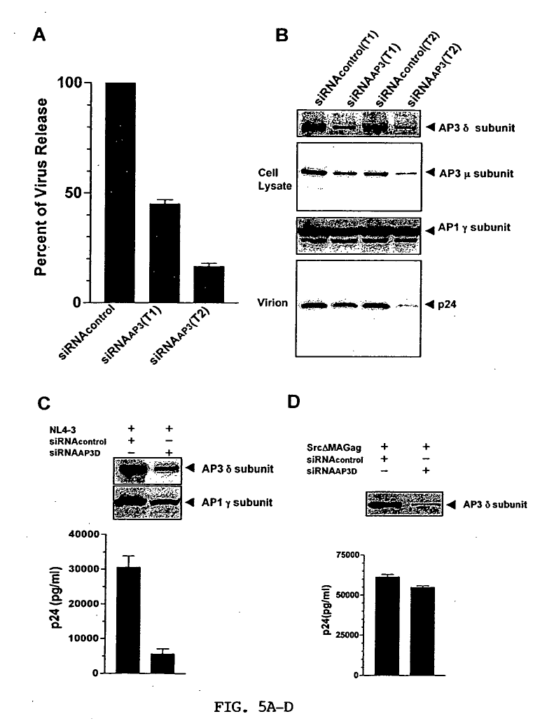

Depletion of the AP-3 Complex Inhibits HIV Particle Assembly. To further define the role of the AP-3 complex in HIV particle assembly, the inventors depleted the cellular AP-3 complex from 293T or Hela cells using siRNA techniques, and evaluated the release of HIV particles from these cells. To achieve a significant depletion of the AP-3 complex in 293T cells, the inventors expressed siRNA for both the δ and μ subunits of the complex, and transfected 293T cells with the combined siRNA at two timepoints. Control experiments with RNA duplexes matched for GC content were included in each experiment. In cells cotransfected with pNL4-3 following a single treatment with siRNA, particle release was inhibited by more than 50% as compared with cells receiving control siRNA (FIG. 5A, T1). A second treatment with siRNA resulted in enhanced depletion of the μ and δ subunits, and particle release was inhibited by more than 80% as compared with transfection of control siRNA (FIG. 5A, T2). The progressive depletion of particle release corresponded to enhanced depletion of endogenous AP-3 components seen in cell lysates, while the AP-1 γ subunit was unaffected (FIG. 5B). The inventors were able to deplete the AP-3 δ subunit more completely following a single transfection of siRNA in Hela cells than in 293T cells. A marked and reproducible inhibition of particle release was demonstrated in Hela cells following a single siRNA transfection targeting only the δ subunit (FIG. 5C). As an additional control for the specificity of this effect, the inventors expressed a myristylated Gag molecule in which MA has been deleted (SrcΔMAGag). The release of SrcΔMAGag was not significantly diminished following siRNA-mediated depletion of the AP-3 delta subunit in Hela cells (FIG. 5D). These results indicate that the Gag-AP-3 δ subunit interaction mediates a productive pathway in particle assembly, and suggest that the v-src myristylation signal in the context of MA-deleted Gag allows Gag to bypass the AP-3 trafficking pathway.

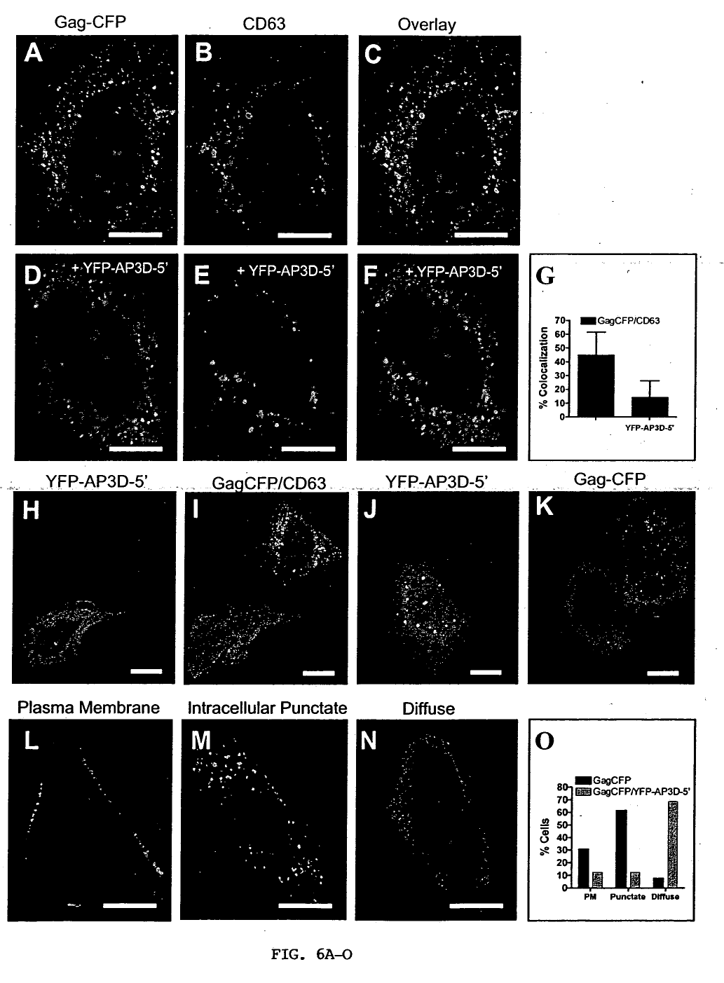

The Gag-AP3 interaction is required for Gag trafficking to MVBs. The AP-3 adaptor complex is known to direct the intracellular trafficking of protein components of the MVB such as CD63 (Dell'Angelica et al., 1999; Pelchen-Matthews et al., 2003; Rous et al., 2002). The inventors hypothesized that the trafficking of Gag to MVBs may similarly be under the control of AP-3. To test this hypothesis, they analyzed the subcellular distribution of Gag-CFP and endogenous CD63 in the presence or absence of the dominant-negative YFP-tagged AP3D-5′ fragment. When analyzed at early timepoints in Hela cells (12-14 hrs post-transfection), a significant fraction of Gag-CFP expressed alone was found in intracellular, punctate sites (FIG. 6A). There was significant colocalization seen with Gag-CFP and endogenous CD63 (FIGS. 6A-C). In the majority of cells in which Gag-CFP was co-expressed with YFP-AP3D-5′, Gag was found in a diffuse cytoplasmic distribution (FIGS. 6J-6K). However, even in those cells expressing YFP-AP3D-5′ that retained a punctate pattern of Gag (FIG. 6D), colocalization with CD63 was not observed (FIG. 6F). The pattern of endogenous CD63 remained predominantly intracellular and punctate in all cells expressing YFP-AP3D-5′ (represented by FIGS. 6E and 6I). The percentage of colocalization of Gag-CFP to CD63 was calculated using the Metamorph software package in cells that did or did not express YFP-AP3D-5′. FIG. 6G demonstrates that Gag-CD63 colocalization was prominent in cells expressing Gag-CFP alone, but was significantly disrupted by the expression of AP3D-5′. The expression of a YFP-tagged dominant-negative AP-3 fragment allowed us to identify cells that differed dramatically in expression of this molecule within the same microscopic field. FIGS. 6H and 6I demonstrate a pair of cells expressing Gag-CFP, of which only the bottom cell expresses the inhibitor. Colocalization of endogenous CD63 and Gag-CFP is observed in the upper cell (FIG. 6I, an overlay of Gag-CFP and CD63 images), while no colocalization is apparent in the cell expressing the inhibitor (FIG. 6I, lower cell).

Next, the inventors asked if Gag was prevented from trafficking to the plasma membrane in cells expressing AP3D-5′. To address this, cells were transfected with Gag-CFP with YFP-AP3D-5′ and examined for Gag-CFP distribution at a later timepoint (20 hours post-transfection). FIGS. 6J and 6K present a pair of cells with differential expression of YFP-AP3D-5′ examined at 20 hours post-transfection. Note that the cell expressing the dominant-negative inhibitor (the lower cell) exhibits a more diffuse, cytoplasmic pattern of Gag than the upper cell, which retains a punctate pattern. This is representative of the predominant pattern seen by confocal microscopy at this timepoint. In order to apply quantitative methods to this observation, the inventors first identified three patterns of Gag-CFP seen within the population of cells. FIG. 6L represents the plasma membrane distribution pattern, FIG. 6M represents the intracellular punctate distribution pattern, and FIG. 6N represents the intracellular diffuse pattern. The inventors then enumerated each pattern from images taken of cells expressing Gag-CFP alone or in the presence of YFP-AP3-5′. FIG. 6O demonstrates that the punctate and plasma membrane pattern of Gag were prominent in cells expressing Gag-CFP alone, while the diffuse pattern was the dominant phenotype in cells expressing the dominant-negative inhibitor. The inventors noted that a subset of cells exhibited a plasma membrane distribution of Gag even in the presence of the inhibitor, suggesting either incomplete inhibition of the AP-3 interaction in these cells or that alternative pathways exist that can lead to Gag-plasma membrane association. Overall, the major conclusions from imaging data were 1) disruption of the Gag-AP-3 interaction eliminates Gag-CD63 colocalization, and 2) AP3D-5′ disrupts both punctate intracellular and plasma membrane patterns of Gag and elicits a diffuse cytoplasmic distribution pattern.

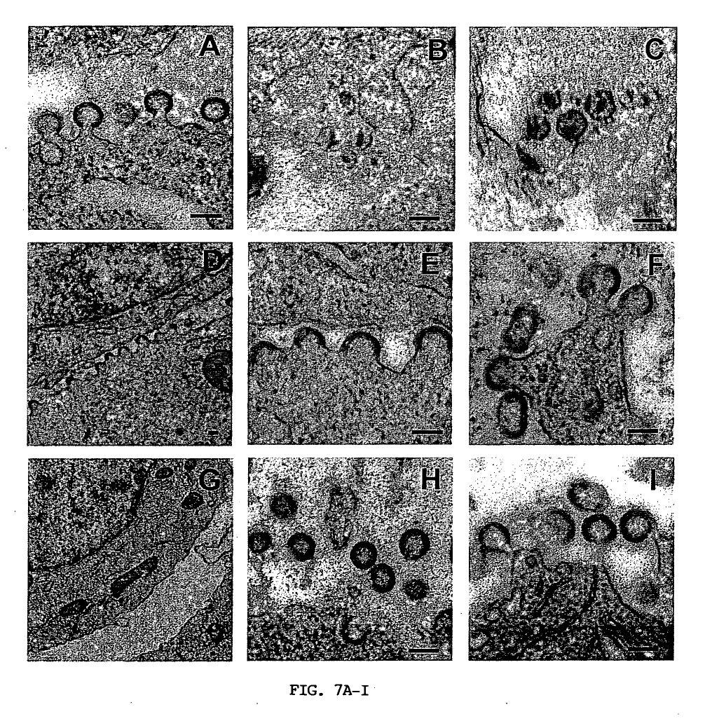

Dominant-negative inhibition of the Gag-AP-3 interaction creates an assembly block distinct from that of TSG-5′. Disruption of the interaction between Gag and TSG101 induced by a truncated molecule (TSG-5′) leads to a late defect in assembly, manifested by numerous arrested particle buds at the plasma membrane of 293T cells (Demirov et al., 2002). The inventors used transmission electron microscopy to define the stage of the block to assembly introduced by AP3D-5′ and compared it with TSG-5′. Normal particle budding was readily observed in 293T cells expressing the NL4-3 provirus in control experiments (FIGS. 7A-C). The inventors noted that in some sections, even upon expression of wild-type NL4-3 alone, tethered particles could be seen in the absence of adjacent mature particles (FIG. 7A). However, in the majority of sections in which retroviral particles were apparent, mature virions that had separated from the plasma membrane could be identified (FIGS. 7B-C). In contrast, cells transfected with pNL4-3 and TSG-5′ demonstrated prominent particle buds that were arrested at the plasma membrane, as previously reported for a late budding defect (FIGS. 7D-F). Abnormally arrested budding particles, including linked double particle shells, were readily apparent (FIG. 7F), and mature virions were less prominent. In cells transfected with pNL4-3 and AP3D-5′ at a 1:1 ratio, very few budding particles were observed. FIG. 7G shows a low-power view of a cell taken from this preparation with an intact plasma membrane and no budding particles, in order to emphasize that this was the predominant observation from analysis of hundreds of examined fields in four separate experiments. In order to provide some quantitation of this observation, the inventors examined 100 consecutive cells prepared for thin-section electron microscopy expressing NL4-3 alone, NL4-3 with TSG-5′, or NL4-3 with AP3D-5′. Of 100 consecutive cells counted, the inventors observed 10 cells with obvious particle budding in pNL4-3-transfected preparations, 12 in cells expressing NL4-3 and TSG-5′ at a 1:1 ratio, and 0 cells demonstrating particle budding from preparations expressing NL4-3 and AP3D-5′. The inventors were able to find rare cells demonstrating particle budding in cells expressing NL4-3 and AP3D-5′ only after examining several hundred additional fields of cells (FIGS. 7H-I). Particle budding was similarly extremely rare in cells in which AP-3 complex was depleted using siRNA (data not shown). In the cells expressing AP3D-5′ that did demonstrate budding, an immature particle morphology was apparent (FIG. 7H) and some evidence of tethering of particle buds was noted (FIG. 7I). The inventors interpret the paucity of observed particle budding events to mean that the block to particle assembly introduced by AP3D-5′ occurs predominantly at a step preceding particle budding. This would be consistent with the predominance of the diffuse cytoplasmic distribution of Gag seen by fluorescence microscopy. The particles that do form buds at the plasma membrane in the presence of AP3D-5′ may exhibit a late defect (FIG. 7I), although the rarity of particles in this case makes the evidence for a late defect much less convincing than for TSG-5′. Taken together with confocal imaging data, these electron microscopy results point to a block that occurs predominantly at a step preceding viral budding.

Cells genetically deficient in AP-3 are resistant to HIV particle production. To follow up on the studies in mammalian cells in which AP-3 depletion or dominant-negative inhibition resulted in a defect in HIV assembly, human cells that are genetically deficient in AP-3 function were studied. Hermansky-Pudlak Syndrome Type II (HPS2) is a rare autosomal recessive disorder characterized by bleeding abnormalities, immune dysfunction, and pigmentary changes that result from a deficiency of AP-3 complex function. The genetic defects responsible for this phenotype map to alterations of the beta subunit of AP-3, either through production of a defective subunit or diminished or absent production of the subunit. As a result, there is a global defect in AP-3-mediated trafficking events in the cell.

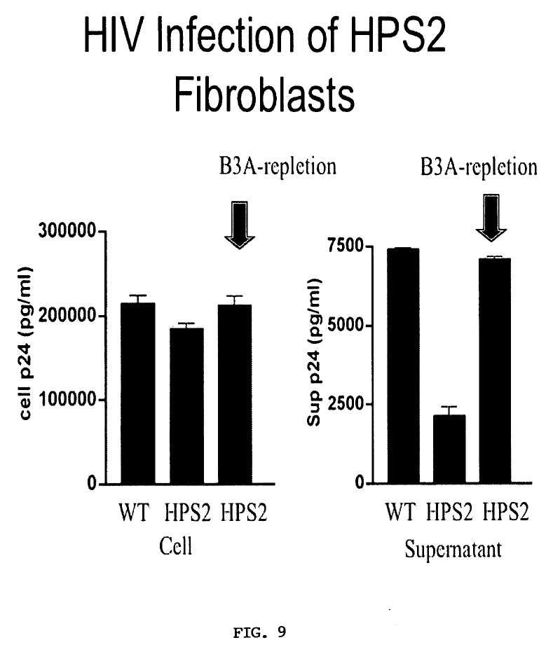

Fibroblasts from HPS2 patients were obtained and were infected with VSV-G-pseudotyped HIV. The production of HIV particles, as monitored by p24 output or by Western blot for released p24, was markedly diminished in HPS2 fibroblasts as compared with normal human fibroblasts. Using retroviral transduction, the defective subunit was replaced in a cell line derived from HPS2 fibroblasts. This intervention restored p24 (particle) output to levels seen with normal human fibroblasts (FIG. 9). These studies provide strong confirmatory evidence that the AP-3 complex is involved in HIV assembly, and support the previous finding that Gag trafficking is abnormal in cells in which AP-3 complex function is diminished or eliminated.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

IX. References

The following references, to the extent that they provide exemplary procedural or other details supplementary to those set forth herein, are specifically incorporated herein by reference.
U.S. Pat. No. 4,166,452
U.S. Pat. No. 4,196,265
U.S. Pat. No. 4,256,108
U.S. Pat. No. 4,265,874
U.S. Pat. No. 4,415,732
U.S. Pat. No. 4,458,066
U.S. Pat. No. 4,946,778
U.S. Pat. No. 5,354,855
U.S. Pat. No. 5,795,715
U.S. Pat. No. 5,888,773
U.S. Pat. No. 5,889,136
Ahle and Ungewickell, J. Biol. Chem. 264:20089-20093, 1989.
Angel et al., Mol. Cell. Biol., 7:2256, 1987.
Angel et al., Cell, 49:729, 1987b.
Angel et al., Mol. Cell. Biol., 7:2256, 1987a.
Atchison and Perry, Cell, 46:253, 1986.
Atchison and Perry, Cell, 48:121, 1987.
Babst et al., Traffic, 1:248-258, 2000.
Baichwal and Sugden, In: Gene Transfer, Kucherlapati (Ed.), NY, Plenum Press, 117-148, 1986.
Banerji et al., Cell, 27(2 Pt 1):299-308, 1981.
Banerji et al., Cell, 33(3):729-740, 1983.
Benvenisty and Neshif, Proc. Natl. Acad. Sci. USA, 83:9551-9555, 1986.
Berkhout et al., Cell, 59:273-282, 1989.
Berlioz-Torrent et al., J. Virol., 73:1350-1361, 1999.
Blanar et al., EMBO J, 8:1139, 1989.
Bodine and Ley, EMBO J, 6:2997, 1987.
Boehm and Bonifacino, Gene, 286:175-186, 2002.
Boge et al., J. Biol. Chem., 273:15773-15778, 1998.
Boshart et al., Cell, 41:521, 1985.
Bosher et al., Nat. Cell. Biol., 2(2):E31-E36, 2000.
Bosze et al., EMBO J, 5(7):1615-1623, 1986.
Braddock et al., Cell, 58:269, 1989.
Bryant and Ratner, Proc. Natl. Acad. Sci. USA, 87:523-527, 1990.
Bulla and Siddiqui, J. Virol., 62:1437, 1986.
Campbell and Villarreal, Mol. Cell. Biol., 8:1993, 1988.
Campbell, In: Monoclonal Antibody Technology, Laboratory Techniques in Biochemistry and Molecular Biology, Burden and Von Knippenberg (Eds.), 13:75-83, Amsterdam, Elseview, 1984.
Campere and Tilghman, Genes and Dev., 3:537, 1989.
Campo et al., Nature, 303:77, 1983.
Caplen et al., Gene, 252:95-105, 2000.
Celander and Haseltine, J. Virology, 61:269, 1987.
Celander et al., J. Virology, 62:1314, 1988.
Chandler et al., Cell, 33:489, 1983.
Chang et al., Hepatology, 14:124A, 1991.
Chang et al., Mol. Cell. Biol., 9:2153, 1989.
Chatterjee et al., Proc. Natl. Acad. Sci. USA, 86:9114, 1989.
Chen and Okayama, Mol. Cell Biol., 7:2745-2752, 1987.
Chien et al., Proc. Natl. Acad. Sci. USA, 88(21):9578-9582, 1991.
Choi et al., Cell, 53:519, 1988.
Clark et al., Nat. Immunol., 4:1111-1120, 2003.
Coffin, In: Virology, Fields et al. (Eds.), Raven Press, NY, 1437-1500, 1990.
Cohen et al., J. Cell. Physiol., 5:75, 1987.
Cohen, J. Natl. Cancer Inst., 90:876-879, 1998.
Colas et al., Nature (London), 380:548-550, 1996.
Collins et al., Cell, 109:523-535, 2002.
Cook et al., Cell, 27:487-496, 1981.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J, 6:3745, 1987.
Culotta and Hamer, Mol. Cell. Biol., 9:1376, 1989.
Dandolo et al., J Virology, 47:55-64, 1983.
Darsow et al., Mol. Biol. Cell, 12:37-51, 2001.
De Villiers et al., Nature, 312(5991):242-246, 1984.
Dell'Angelica et al., EMBO J, 16:917-928, 1997.
Dell'Angelica et al., J. Biol. Chem., 275:1300-1306, 2000.
Dell'Angelica et al., Mol. Cell, 3:11-21, 1999.
Demirov et al., Proc. Natl. Acad. Sci. USA, 99:955-960, 2002.
Derdowski et al., J. Virol., 78:1230-1242, 2004.
Deschamps et al., Science, 230:1174-1177, 1985.
Dubensky et al., Proc. Natl. Acad. Sci. USA, 81:7529-7533, 1984.
Edbrooke et al., Mol. Cell. Biol., 9:1908, 1989.
Edlund et al., Science, 230:912-916, 1985.
Elbashir et al., EMBO, 20(23):6877-6888, 2001.
Eur. Pat. App. No. 0273085
Fabbrizio et al., Oncogene, 18(30):4357-4363, 1999.
Facke et al., J. Virol., 67:4972-4980, 1993.
Fearon et al., Proc. Natl. Acad. Sci. USA, 89(17):7958-7962, 1992.
Fechheimer, et al., Proc. Natl. Acad. Sci. USA, 84:8463-8467, 1987.
Feng and Holland, Nature, 334:6178, 1988.
Ferkol et al., FASEB J., 7:1081-1091, 1993.
Firak and Subramanian, Mol. Cell. Biol., 6:3667, 1986.
Fire et al., Nature, 391:806-811, 1998.
Foecking and Hofstetter, Gene, 45(1):101-105, 1986.
Forster and Symons, Cell, 49:211-220, 1987.
Fraley, et al., Proc Natl. Acad. Sci. USA, 76:3348-3352, 1979.
Franke et al., Nature, 372:359-362, 1994.
Freed and Martin, J. Virol., 70:341-351, 1996.
Freed et al., J. Virol., 68:5311-5320, 1994.
Freed, Virology, 251:1-15, 1998.
Friedmann, Science, 244:1275-1281, 1989.
Fujita et al., Cell, 49:357, 1987.
Garrus et al., Cell, 107:55-65, 2001.
Gefter, et al., Somatic Cell Genet., 3:231-236, 1977.
Gerlach et al., Nature (London), 328:802-805, 1987.
Geyer et al., Proc. Natl. Acad. Sci. USA, 96:8567-8572, 1999.
Ghosh and Bachhawat, In: Liver Diseases, Targeted Diagnosis and Therapy Using Specific Receptors and Ligands. Wu et al. (Eds.), Marcel Dekker, NY, 87-104, 1991.
Ghosh-Choudhury et al., EMBO J, 6:1733-1739, 1987.
Gilles et al., Cell, 33:717, 1983.
Gloss et al., EMBO J., 6:3735, 1987.
Godbout et al., Mol. Cell. Biol., 8:1169, 1988.
Goding, In: Monoclonal Antibodies: Principles and Practice, 2^{nd} Ed., Academic Press, Fl, 60-61, and 71-74, 1986.
Gomez-Foix et al, J. Biol. Chem., 267:25129-25134, 1992.
Goodbourn and Maniatis, Proc. Natl. Acad. Sci. USA, 85:1447, 1988.
Goodbourn et al., Cell, 45:601, 1986.
Goodman and Gilman's The Pharmacological Basis Of Therapeutics, Hardman et al. (Eds.), 10^{th} Ed., 32:853-860; 35:891-893, 2001.
Gottlicher et al., EMBO J, 20:6969-78, 2001.
Gottlinger et al., Proc. Natl. Acad. Sci. USA, 86:5781-5785, 1989.
Gould et al., Proc. Natl. Acad. Sci. USA, 100, 10592-10597, 2003.
Graham and Prevec, In: Methods in Molecular Biology: Gene Transfer and Expression Protocol, Murray (Ed.), Humana Press, Clifton, N.J., 7:109-128, 1991.
Graham and van der Eb, Virology, 52:456-467, 1973.
Graham et al., J. Gen. Virol., 36:59-72, 1977.
Greene et al., Immunology Today, 10:272, 1989
Grishok et al., Science, 287:2494-2497, 2000.
Grosschedl and Baltimore, Cell, 41:885, 1985.
Grunhaus and Horwitz, Seminar in Virology, 3:237-252, 1992.
Gyuris et al., Cell, 75(4):791-803, 1993.
Harland and Weintraub, J. Cell Biol., 101:1094-1099, 1985.
Harlow and Lane, Antibodies: A Laboratory manual, Cold Spring Harbor Laboratory, 1988.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Natl. Acad. Sci. USA, 90:2812-2816, 1993.
Hill et al., Proc. Natl. Acad. Sci. USA, 93:3099-3104, 1996.
Hirochika et al., J. Virol., 61:2599, 1987.
Hirsch et al., Mol. Cell. Biol., 10:1959, 1990.
Holbrook et al., Virology, 157:211, 1987.
Hoppe-Seyler et al., J Steroid Biochem. Mol. Biol., 78:105-111, 2004.
Horlick and Benfield, Mol. Cell. Biol., 9:2396, 1989.
Horwich, et al., J. Virol., 64:642-650, 1990.
Huang et al., Cell, 27:245, 1981.
Huang et al., J. Virol., 75:4947-4951, 2001.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Janvier et al., J. Biol. Chem., 278:8725-8732, 2003a.
Janvier et al., J. Cell Biol., 163:1281-1290, 2003b.
Jaynes et al., Mol. Cell. Biol., 8:62, 1988.
Johnson et al., Mol. Cell. Biol., 9:3393, 1989.
Jones and Shenk, Cell, 13:181-188, 1978.
Joyce, Nature, 338:217-244, 1989.
Kadesch and Berg, Mol. Cell. Biol., 6:2593, 1986.
Kaneda et al., Science, 243:375-378, 1989.
Karin et al., Mol. Cell. Biol., 7:606, 1987.
Karin et al., Mol. Cell. Biol., 7:606, 1987.
Karlsson et al., EMBO J, 5:2377-2385, 1986.
Katinka et al., Cell, 20:393, 1980.
Kato et al., J. Biol. Chem., 266(6):3361-3364, 1991.
Katzmann et al., Cell, 106:145-155, 2001.
Kawamoto et al., Mol. Cell. Biol., 8:267, 1988.
Ketting et al., Cell, 99(2):133-141, 1999.
Kiledjian et al., Mol. Cell. Biol., 8:145, 1988.
Kim and Cook, Proc. Natl. Acad. Sci. USA, 84:8788-8792, 1987.
Klamut et al., Mol. Cell. Biol., 10:193, 1990.
Klein et al., Nature, 327:70-73, 1987.
Koch et al., Mol. Cell. Biol., 9:303, 1989.
Kohler and Milstein, Eur. J. Immunol., 6:511-519, 1976.
Kohler and Milstein, Nature, 256:495-497, 1975.
Kolonin and Finley, Proc. Natl. Acad. Sci. USA, 95(24):14266-71, 1998.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
Larsen et al., Proc. Natl. Acad. Sci. USA, 83:8283, 1986.
Laspia et al., Cell, 59:283, 1989.
Latimer et al., Mol. Cell. Biol., 10:760, 1990.
Le Borgne et al., J. Biol. Chem., 273:29451-29461, 1998.
Le Gal La Salle et al., Science, 259:988-990, 1993.
Lee et al., Nature, 294:228, 1981.
Lee et al., Nucleic Acids Res., 12:4191-206, 1984.
Levinson et al., Nature, 295:79, 1982.
Levrero et al., Gene, 101: 195-202, 1991.
Lin et al., Genetics, 153:1245-1256, 1999.
Lin et al., Mol. Cell. Biol., 10:850, 1990.
Lindwasser and Resh, J. Virol., 78:6013-6023, 2004.
Lodge et al., J. Virol., 68:4857-4861, 1994.
Luban, Nat. Med., 7:1278-1280, 2001.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Samow, Nature, 353:90-94, 1991.
Majors and Varmus, Proc. Natl. Acad. Sci. USA, 80:5866, 1983.
Mann et al., Cell, 33:153-159, 1983.
Markowitz et al., J. Virol., 62:1120-1124, 1988.
Martin-Serrano et al., J. Virol., 77:4794-4804, 2003.
Matthews et al., Nature, 370:666-668, 1994.
McDonald et al., Science, 300:1295-1297, 2003.
McNeall et al., Gene, 76:81, 1989.
Michel and Westhof, J. Mol. Biol., 216:585-610, 1990.
Miksicek et al., Cell, 46:203, 1986.
Montgomery et al., Proc. Natl. Acad. Sci. USA, 95:15502-15507, 1998.
Mordacq and Linzer, Genes and Dev., 3:760, 1989.
Moreau et al., Nucl. Acids Res., 9:6047, 1981.
Muesing et al., Cell, 48:691, 1987.
Nakatsu and Ohno, Cell Struct. Funct., 28:419-429, 2003.
Ng et al., Nuc. Acids Res., 17:601, 1989.
Nguyen et al., J. Biol. Chem., 278:52347-52354, 2003.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nydegger et al., Traffic, 4:902-910, 2003.
Ohno et al., Virology, 238:305-315, 1997.
Ondek et al., EMBO J., 6:1017, 1987.
Ono and Freed, J. Virol., 78:1552-1563, 2004.
Ono et al., J. Virol., 74:2855-2866, 2000.
Ornitz et al., Mol. Cell. Biol., 7:3466, 1987.
Palmiter et al., Nature, 300:611, 1982.
Paskind et al., Virology, 67:242-248, 1975.
PCT Appln. WO 00/44914.
PCT Appln. WO 01/36646.
PCT Appln. WO 01/68836.
PCT Appln. WO 84/03564.
PCT Appln. WO 99/32619.
Pech et al., Mol. Cell. Biol., 9:396, 1989.
Peden et al., J. Cell Biol., 164:1065-1076, 2004.
Pelchen-Matthews et al., J. Cell Biol., 162:443-455, 2003.
Pelletier and Sonenberg, Nature, 334:320-325, 1988.
Perales, et al., Proc. Natl. Acad. Sci. USA, 91(9):4086-4090, 1994.
Perez-Stable and Constantini, Mol. Cell. Biol., 10:1116, 1990.
Pevsner et al., Gene, 146:279-283, 1994.
Physicians Desk Reference.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Pornillos et al., J. Cell Biol., 162:425-434, 2003.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotechnology Techniques, 9:169-174, 1995.
Ragot et al., Nature, 361:647-650, 1993.
Raposo et al., Traffic, 3:718-729, 2002.
Redondo et al., Science, 247:1225, 1990.
Rehling et al., Nat. Cell Biol., 1:346-353, 1999.
Reil et al., EMBO J, 17:2699-2708, 1998.
Reinhold-Hurek and Shub, Nature, 357:173-176, 1992.
Reisman and Rotter, Mol. Cell. Biol., 9:3571, 1989.
Remington's Pharmaceutical Sciences, 15^{th} ed., 1035-1038 and 1570-1580, Mack Publishing Company, Easton, Pa., 1980.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A Survey of Molecular Cloning Vectors and Their Uses, Rodriguez et al. (Eds), Stoneham: Butterworth, 467-492, 1988.
Ripe et al., Mol. Cell. Biol., 9:2224, 1989.
Rippe, et al., Mol. Cell Biol., 10:689-695, 1990.
Rittling et al., Nuc. Acids Res., 17:1619, 1989.
Robinson and Bonifacino, Curr. Opin. Cell Biol., 13:444-453, 2001.
Rosen et al., Cell, 41:813, 1988.
Rosenfeld et al., Science, 252:431-434, 1991.
Rosenfeld, et al., Cell, 68:143-155, 1992.
Rous et al., Mol. Biol. Cell, 13:1071-1082, 2002.
Roux et al., Proc. Natl. Acad. Sci. USA, 86:9079-9083, 1989.
Sakai et al., Genes and Dev., 2:1144, 1988.
Sandefur et al., J. Virol., 74:7238-7249, 2000.
Sarver et al., Science, 247:1222-1225, 1990.
Satake et al., J Virology, 62:970, 1988.
Scanlon et al., Proc. Natl. Acad. Sci. USA, 88:10591-10595, 1991.
Schafffier et al., J. Mol. Biol., 201:81, 1988.
Schneider et al., Biotechniques, 20:960-962, 1996.
Searle et al., Mol. Cell. Biol., 5:1480, 1985.
Sharp and Marciniak, Cell, 59:229, 1989.
Sharp et al., Science, 287:2431-2433, 2000.
Sharp, P. A., Genes. Dev., 13:139-141, 1999.
Shaul and Ben-Levy, EMBO J, 6:1913, 1987.
Sherer et al., Traffic, 4:785-801, 2003.
Sherman et al., Mol. Cell. Biol., 9:50, 1989.
Shih et al., J. Biol. Chem., 270:31083-31090, 1995.
Simpson et al., J. Cell Biol., 133:749-760, 1996.
Simpson et al., J. Cell Biol., 137:835-845, 1997.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Spearman et al., J. Virol., 68:3232-3242, 1994.
Starcevic et al., Semin. Cell Dev. Biol., 13:271-278, 2002.
Stephens and Hentschel, Biochem. J., 248:1, 1987.
Strack et al., Cell, 114:689-699, 2003.
Stratford-Perricaudet and Perricaudet, In: Human Gene Transfer, Cohen-Haguenauer et al., (Eds.), John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Tabara et al., Cell, 99(2): 123-132, 1999.
Takebe et al, Mol. Cell. Biol., 8:466, 1988.
Tavernier et al., Nature, 301:634, 1983.
Taylor and Kingston, Mol. Cell. Biol., 10: 165, 1990a.
Taylor and Kingston, Mol. Cell. Biol., 10: 176, 1990b.
Taylor et al., J. Biol. Chem., 264:15160, 1989.
Temin, In: Gene Transfer, Kucherlapati (Ed.), NY, Plenum Press, 149-188, 1986.
The Merck Index, O'Neil et al. (Ed.), 13^{th} Ed., 2001.
Thiesen et al., J. Virology, 62:614, 1988.
Top et al., J. Infect. Dis., 124:155-160, 1971.
Treisman, Cell, 42:889, 1985.
Tronche et al., Mol. Biol. Med., 7:173, 1990.
Trudel and Constantini, Genes and Dev. 6:954, 1987.
Tur-Kaspa, et al., Mol. Cell Biol., 6:716-718, 1986.
Tyndell et al., Nuc. Acids. Res., 9:6231, 1981.
Vannice and Levinson, J. Virology, 62:1305, 1988.
Varmus et al., Cell, 25:23-36, 1981.
Vasavada et al., Proc. Natl. Acad. Sci. USA, 88(23):10686-10690, 1991.
Vasseur et al., Proc. Natl. Acad. Sci. USA, 77:1068, 1980.
VerPlank et al., Proc. Natl. Acad. Sci. USA, 98:7724-7729, 2001.
von Schwedler et al., Cell, 114:701-713, 2003.
Wagner, et al., Proc. Natl. Acad. Sci. USA, 87(9):3410-3414, 1990.
Wang and Calame, Cell, 47:241, 1986.
Weber et al., Cell, 36:983, 1984.
Weinberger et al. Mol. Cell. Biol., 8:988, 1984.
Wills and Craven, AIDS, 5:639-654, 1991.
Wincott et al., Nucleic Acids Res., 23:2677-2684, 1995.
Winoto and Baltimore, Cell, 59:649, 1989.
Wodrich et al., Nucleic Acids Res., 28:901-910, 2000.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Xu et al., Proc. Natl. Acad. Sci., 94:12473-12478, 1997.
Yang, et al., Proc Natl. Acad. Sci. USA, 87:9568-9572, 1990.
Yu et al., J. Virol., 66:4966-4971, 1992.
Yuan et al., J. Virol., 67:6387-6394, 1993.
Yutzey et al. Mol. Cell. Biol., 9:1397, 1989.
Zelenin et al., FEBS Lett., 280:94-96, 1991.
Zhou and Resh, J. Virol., 70:8540-8548, 1996.

What is claimed is:

1. A method of screening a first substance for anti-HIV activity comprising:
(a) providing δ subunit of adaptor protein-3 (δAP-3) and Gag under conditions permitting the formation of an δAP-3/Gag complex;
(b) contacting said first substance with δAP-3 and Gag; and
(c) assessing the formation of δAP-3/Gag complex;
wherein a decrease in the amount of δAP-3/Gag complex, as compared to δAP-3/Gag complex formed in the absence of said first substance, indicates that said first substance possesses anti-HIV activity.

2. The method of claim 1, wherein step (a) comprises providing a cell that expresses δAP-3 and Gag.

3. The method of claim 2, wherein step (a) comprises providing a cell infected with HIV.

4. The method of claim 1, wherein step (a) comprises providing δAP-3 and Gag in a cell free environment.

5. The method of claim 1, wherein step (c) comprises a two-hybrid screen.

6. The method of claim 1, wherein step (c) comprises a Western blot.

7. The method of claim 1, wherein step (c) comprises a band shift assay.

8. The method of claim 1, wherein step (c) comprises a sandwich ELISA.

9. The method of claim 1, wherein step (c) comprises co-immunoprecipitation.

10. The method of claim 1, further comprising performing a control reaction wherein a known inhibitor of δAP-3/Gag complex formation is used.

11. The method of claim 1, further comprising performing a control reaction wherein no inhibitor of δAP-3/Gag complex formation is used.

12. The method of claim 1, wherein said first substance is a protein or peptide.

13. The method of claim 1, wherein said first substance is a nucleic acid.

14. The method of claim 13, wherein said nucleic acid is an antisense molecule, a ribozyme or a small interfering RNA.

15. The method of claim 1, wherein said first substance is a small molecule.

16. The method of claim 1, further comprising the addition of a second substance distinct from said first substance.

17. A method of screening a first substance for anti-HIV activity comprising:
(a) providing a cell that expresses δ subunit of adaptor protein-3 (δAP-3);
(b) contacting said cell with said first substance; and
(c) assessing the expression of δAP-3;
wherein a decrease in the amount of δAP-3, as compared to the δAP-3 expressed in the absence of said first substance, indicates that said first substance possesses anti-HIV activity.

18. The method of claim 17, further comprising assessing the effect of said first substance on δAP-3/Gag complex formation.

19. The method of claim 17, further comprising assessing the effect of said first substance on HIV particle formation.

20. The method of claim 17, wherein assessing comprises radioimmune precipitation, immunoblot, ELISA, RIA, or quantitive RT-PCR.

21. The method of claim 17, wherein said first substance is a protein, a peptide, a nucleic acid or a small molecule.

22. A method of inhibiting HIV infection comprising contacting a subject infected or suspected of being infected with HIV with a substance that inhibits formation of δ subunit of AP-3(δAP-3)/Gag complex.

23. The method of claim 22, wherein said substance is an δAP-3 antisense molecule.

24. The method of claim 22, wherein said substance is an δAP-3 siRNA molecule.

25. The method of claim 22, wherein said substance is an anti-δAP-3 antibody molecule.

26. The method of claim 22, wherein said substance is a small molecule or protein.

27. The method of claim 22, wherein said substance is a dominant negative form of δAP-3.

28. The method of claim 22, wherein said substance is an expression construct encoding an δAP-3 antisense molecule, siRNA, dominant negative form of δAP-3 or anti-δAP-3 antibody.

29. The method of claim 22, further comprising administering a second anti-HIV agent to said subject.

30. The method of claim 29, wherein said second anti-HIV agent is a nucleoside analog or a reverse transcriptase inhibitor.

31. A pharmaceutical formulation comprising (a) an inhibitor of δ subunit of AP-3 expression and (b) a pharmaceutical carrier, buffer, diluent or excipient.


SEQUENCE LISTING (7 sequences)

SEQ ID NO: 1 - DNA, 19 bp, Artificial Sequence
  Description of Artificial Sequence  Synthetic Primer
tctgcaagctgacgtattt

SEQ ID NO: 2 - DNA, 19 bp, Artificial Sequence
  Description of Artificial Sequence  Synthetic Primer
gaagaaggctgtgctgatc

SEQ ID NO: 3 - DNA, 19 bp, Artificial Sequence
  Description of Artificial Sequence  Synthetic Primer
gcgagaaactgcctattca

SEQ ID NO: 4 - DNA, 19 bp, Artificial Sequence
  Description of Artificial Sequence  Synthetic Primer
gaaggacgttcccatggta

SEQ ID NO: 5 - DNA, 19 bp, Artificial Sequence
  Description of Artificial Sequence  Synthetic Primer
ttgagttcctacatcgagt

SEQ ID NO: 6 - RNA, 21 nt, Artificial Sequence
  Description of Artificial Sequence  Synthetic Primer
gcugaguauacgcggauguuu

SEQ ID NO: 7 - RNA, 21 nt, Artificial Sequence
  Description of Artificial Sequence  Synthetic Primer
accaccaacauaucuacgcuu